(19) United States
(12) Patent Application Publication
(10) Pub. No.: US 2026/0218291 A1
(43) Pub. Date: Jul. 30, 2026

(54) METHODS AND SYSTEMS FOR SEQUENCING OF NUCLEIC ACID MOLECULES USING A SINGLE SEQUENCING ASSAY

(71) Applicant: Yale University, New Haven, CT (US)

(72) Inventors: Morgan Levine, New Haven, CT (US); Albert Higgins-Chen, New Haven, CT (US); Raghav Sehgal, New Haven, CT (US); Margarita Meer, New Haven, CT (US)

(21) Appl. No.: 19/400,859

(22) Filed: Nov. 25, 2025

Related U.S. Application Data
(63) Continuation of application No. 19/157,758, filed on Feb. 19, 2024.
(60) Provisional application No. 63/486,023, filed on Feb. 20, 2023.

(51) Int. Cl. C12Q 1/6869; C12Q 1/6806

(52) U.S. Cl. CPC C12Q 1/6869; C12Q 1/6806; C12Q 2600/154

(57) ABSTRACT

Methods and systems are described for sequencing one or more nucleic acid molecules. The methods may include obtaining a biological sample from a subject. The methods may include deriving one or more nucleic acid molecules from the biological sample of the subject. The methods may include sequencing the one or more nucleic acid molecules using a single genomic sequencing assay. The systems may include systems for executing these methods.

### CROSS-REFERENCE TO RELATED APPLICATIONS

The present application is a continuation of, and claims priority to, U.S. patent application Ser. No. 19/157,758, filed Aug. 19, 2025, which is a 35 U.S.C. § 371 national phase application of, and claims priority to, PCT International Patent Application No. PCT/US2024/016361, filed Feb. 19, 2024, which claims priority under 35 U.S.C. § 119 (e) to U.S. Provisional Patent Application No. 63/486,023, filed Feb. 20, 2023, the contents of which applications are incorporated herein by reference in their entireties.

### STATEMENT REGARDING FEDERALLY SPONSORED RESEARCH OR DEVELOPMENT

This invention was made with government support under 1R01AG065403 and 5R01AG060110 awarded by the National Institutes of Health. The government has certain rights in the invention.

### BACKGROUND OF THE INVENTION

The geroscience hypothesis states that directly targeting the biology of aging can improve human health and delay the onset of multiple chronic diseases simultaneously by slowing biological aging. Yet, to truly test this hypothesis, reliable biomarkers must be developed that reflect valid age-related changes and responses to interventions. “Epigenetic clocks”, based on DNA methylation (DNAm), are among the most studied aging biomarkers to date. Multiple versions have been developed, some to predict different age-related outcomes. Most utilize information on a few hundred CpGs to report a single biological age value for each individual that is meant to reflect how the individual's degree of biological aging compares to a reference population. For a number of epigenetic clocks, discordance between predicted and observed age has been shown to be biologically meaningful, as it is predictive of age-related morbidity and mortality, and correlates with other age-related phenotypes. More recently, DNAm markers have been built to predict longitudinal changes in clinical indicators of aging, referred to as pace of aging, and these too show associations with age-related health outcomes.

Existing DNAm clocks report an individual's overall degree or pace of aging as a single value, capturing the heterogeneity between individuals. However, there is also heterogeneity in the aging process within individuals, at various levels of biological organization. For instance, there is variation in the rate of aging between organ systems, organs, tissues, and even cells. Existing blood-based DNAm clocks do not address system-specific differences in aging. Though blood methylation is utilized in numerous aging studies due to ease of access, it remains unclear how much information about various other organ systems can be gleaned from blood methylation alone. Attempts have been made to build DNAm biomarkers that capture some cardiac or metabolic disease risk, though they tend to only be targeted to a single system. Another important caveat is that physiological systems can function or decline independently of each other, as well as in concert through their interactions. This gives rise to a third type of heterogeneity in aging-heterogeneity in aging that manifests in the overall pattern of decline across physiological systems. This is important for geroscience applications and for the prevention of multimorbidity by targeting biological aging, as diseases are often caused by a combination of malfunctioning in specific biological systems. For example, arthritis involves musculoskeletal deterioration and inflammation; while stroke may be caused by a combination of cardiovascular, metabolic, inflammatory, and neurological factors. These patterns across physiological systems may give rise to aging subtypes that predispose an individual to a subset of specific aging conditions.

Failing to capture the different levels of heterogeneity in the aging process has practical consequences for the assessment of aging patients and populations. Two individuals can have different DNAm profiles that produce the exact same epigenetic age as calculated by blood-based epigenetic clocks, yet they may be physiologically deteriorating in entirely different systems. Additionally, two individuals may have the exact same age as calculated by blood-based methylation clocks, yet they may be predisposed to different diseases depending on the co-occurrence of aging across other systems in their body. As such, there remains a need in the art for improved methods of epigenetic measurement that improve on existing epigenetic clocks by providing system-level measurements. The present invention addresses this need.

### SUMMARY OF THE INVENTION

In one aspect a method of training an algorithm to determine systems-level epigenetic scores includes grouping biomarkers from a first dataset including biomarker data and DNA methylation data into biological systems; generating eigenvector matrices from the first dataset, the generating of the eigenvector matrices comprising performing principal component analysis (PCA) on the biomarkers, creating biomarker principal component (PC) scores for each individual in the dataset and performing PCA on the DNA methylation data from the first dataset, creating DNA methylation PC scores for each individual in the dataset; inputting the DNA methylation PC scores into a supervised elastic net penalized regression, generating a model including system PC predictors for each of the biological systems; applying the eigenvector matrices to a second dataset including DNA methylation data and linked mortality data, generating estimated DNA methylation PC scores for each individual in the second dataset; inputting the estimated DNA methylation PC scores into the model, producing DNA methylation proxies for system-specific PC scores; and separately for each of the biological systems, inputting the DNA methylation proxies of system-specific PC scores into a Cox elastic net penalized regression, generating a system-specific epigenetic age predictor for each of the biological systems.

In some embodiments, the biomarker data in the first dataset includes clinical chemistry assays measured in plasma and serum; physiological measurements; functional tests; and history of symptoms and diseases. In some embodiments, the eigenvector matrices reduce dimensionality and remove collinearity. In some embodiments, the biological systems comprise at least 11 different systems. In some embodiments, the biological systems include blood, brain, cardiac, hormone, immune, inflammation, kidney, liver, lung, metabolic, and musculoskeletal.

In some embodiments, the biomarkers of the blood system comprise Ferritin, Hematocrit, Hemoglobin, Mean Corpuscular Hemoglobin, Mean Corpuscular Hemoglobin Concentration, Mean Corpuscular Volume, Mean Platelet Volume, Platelet Distribution Width, Platelet Count, Red Blood Cell Count, and Red Cell Distribution Width.

In some embodiments, the biomarkers of the brain system comprise Homocysteine, Serum BDNF, Clusterin, total mental status summary score, total cognition summary score, immediate word recall score, delayed word recall score, total word recall summary score, serial 7s test score, and history of stroke.

In some embodiments, the biomarkers of the cardiac system comprise Homocysteine, BMI, systolic blood pressure, diastolic blood pressure, waist circumference, pulse, history of shortness of breath while awake, and PC components of GrimAge.

In some embodiments, the biomarkers of the hormone system comprise Dehydroepiandrosterone sulphate and IGF1.

In some embodiments, the biomarkers of the immune system comprise Eosinophil Count, Lymphocyte Count, Monocyte Count, Neutrophil Count, Basophils percent, Eosinophils percent, Lymphocytes percent, Monocytes percent, White Blood Cell Count, Myeloid Dendritic cells (DC-M) percent, Plasmacytoid Dendritic Cells (DC-P) percent, NK Cells: CD56HI percent, NK Cells: CD56LO percent, CD16-Monocytes percent, CD16+ Monocytes percent, B Cells percent, CD8+ T Cells: Central Memory (CM) percent, CD4+ T Cells: Central Memory (CM) percent, CD8+ T Cells percent, CD8+ T Cells: (TemRA) percent, CD4+ T Cells: (TemRA) percent, CD4+ T Cells percent, IgD+ Memory B Cells percent, IgD-Memory B Cells percent, CD8+ T Cells: Naive percent, CD4+ T Cells: Naive percent, T Cells percent, Naive B Cells percent, CD8+ T Cells: Effector Memory (Tem) percent, CD4+ T Cells: Effector Memory (Tem) percent, NK Cells percent, Monocytes percent, and Dendritic Cells percent.

In some embodiments, the biomarkers of the inflammation system comprise C-Reactive Protein, Transforming Growth Factor Beta, Interleukin 10, Interleukin 1 Receptor Antagonist, Interleukin 6, Tumor Necrosis Factor Receptor 1, and Ferritin.

In some embodiments, the biomarkers of the kidney system comprise Albumin, Urea Nitrogen, Chloride, Bicarbonate, Creatinine, Cystatin C, Potassium, and Sodium.

In some embodiments, the biomarkers of the liver system comprise Albumin, Alkaline Phosphatase, ALT, AST, Bilirubin, and Total Protein.

In some embodiments, the biomarkers of the lung system comprise Bicarbonate, PC prediction of smoking pack-years, history of chronic lung disease, history of shortness of breath while awake, history persistent wheezing/cough/phlegm, peak expiratory flow, and receiving oxygen.

In some embodiments, the biomarkers of the metabolic system comprise C-Reactive Protein, Glucose-Fasting, HDL-Cholesterol, LDL-Cholesterol, Triglycerides, Interleukin-6, history of Diabetes, BMI, and Waist circumference.

In some embodiments, the biomarkers of the musculoskeletal system comprise Vitamin D3, DHEASE, IGF1, history of arthritis, height, weight, BMI, history of difficulty with mobility, history of back problems, maximum grip strength, grip strength left and right, semi tandem balance test time, full tandem balance test time, side-by-side balance test time, timed walk test time, timed walk test time with walking aid, and difficulty doing daily physical movements. In some embodiments, the daily physical movements comprise stooping/kneeling/crouching, walking one block, walking several blocks, climbing several flights of stairs, climbing one flight of stairs, getting up from a chair, raising arms above one's head, carrying 10 lbs, and picking up a dime.

In some embodiments, the method further includes performing Cox elastic net penalized regression to predict mortality using a combination of the system-specific epigenetic age predictor for each of the biological systems, generating a combined systems age measure. In some embodiments, the combined systems age measure predicts aging phenotypes without a bias towards particular phenotypes.

In another aspect, a method of calculating systems-level epigenetic scores includes applying the algorithm according to claim 1 to a blood sample from a subject; wherein the algorithm calculates epigenetic scores for individual biological systems based upon data derived from the blood sample. In some embodiments, the method further includes calculating a combined systems age measure using the algorithm according to one or more of the embodiments disclosed herein.

In another aspect, an apparatus for calculating systems-level epigenetic scores includes a processor; a memory unit; and a communication interface; wherein the processor is connected to the memory unit and the communication interface; and wherein the processor and memory are configured to implement the method of any one of the embodiments disclosed herein.

In another aspect, provided herein is a computer readable storage medium storing computer-executable instructions for performing the method according to any of the embodiments disclosed herein.

### BRIEF DESCRIPTION OF THE DRAWINGS

For a fuller understanding of the nature and desired objects of the present invention, reference is made to the following detailed description taken in conjunction with the accompanying figures wherein like reference characters denote corresponding parts throughout the several views.

FIG. 1 shows an image illustrating hierarchy of heterogeneity in aging. Heterogeneity in aging starts at the very cellular and subcellular levels due to genetic and environmental factors. These variations in aging go on to accumulate at the tissue, organ and the biological system level causing differences in the rates of aging of different systems within an individual. Of course, these systems do not behave independently of each other and this leads to certain common patterns of deterioration across systems giving rise to aging subtypes. Eventually, all of these variations accumulate at the whole body level to cause variations in overall aging rates across individuals. Most epigenetic aging clocks typically focus on the whole body aging level of heterogeneity. In contrast, Systems Age aims to capture the systems level heterogeneity and aging subtypes (while also maintaining the measurement of whole body aging).

FIG. 2 shows a schematic illustrating an analysis pipeline. Step 1—Grouping Biomarkers into systems; Step 2—Deconvoluting systems into principal components; Step 3—Building DNAm surrogates of system PCs using ElasticNet regression; Step 4—Building system scores by combining system PCs using Cox ElasticNet regression; Step 5—Building Systems Age by combining system scores using Cox ElasticNet regression. Training done in HRS and FHS datasets while testing for specificity and aging subtypes done in WHI.

FIG. 3 shows an image illustrating meta-analysis associations (z-scores calculated using a race stratified analysis of 3 WHI datasets) for specific diseases and aging phenotypes with system score age accelerations depicted with text size and color. The system(s) with the highest positive association (or lowest in case of negative association) is bolded and the organ is colored on the human figure. N for functional phenotypes ranges between 1172 and 5127. For time to disease events and disease prevalence at baseline, total N as well as number of events or individuals with diseases has been provided (in brackets). Total N for the time to disease events and disease prevalence at baseline is typically around 5000. For functional phenotypes at baselines Ordinary Least Squares regression model was used, for time to disease events cox proportional hazard models were used and for disease prevalence at baseline logistic regression models were used. Models built for each racial group separately and then meta-analyzed via a fixed effects model with inverse variance weights. Exact z-scores as well as heterogeneity p-values and other phenotypes are given in Tables 3 and 4.

FIG. 4 shows graphs illustrating meta-analysis associations (z-scores calculated using a race stratified analysis of 3 WHI datasets) for specific diseases and aging phenotypes with age accelerations of different clocks, Systems Age and the best system score plotted for smoking status adjusted (in darker shades) and no smoking status adjusted (lighter shades). N for functional measures ranges between 1172 and 4145. For time to disease events and disease prevalence at baseline, total N as well as number of events or individuals with diseases has been provided (in brackets). Total N for the time to disease events and disease prevalence at baseline is typically around 5000. For functional measures at baseline Ordinary Least Squares regression model was used, for time to disease events cox proportional hazard models were used and for disease prevalence at baseline logistic regression models were used. Models built for each racial group separately and then meta-analyzed via a fixed effects model with inverse variance weights. Exact z-scores as well as heterogeneity p-values are given in Tables 3-6.

FIGS. 5A-D show graphs and images illustrating aging subtypes. (A) Correlations between system scores across all WHI cohorts corrected for batch effects (N=5129). Exact correlations provided in Table 9. (B) Three chronological age matched individuals with the same race and gender as well as similar age-accelerated Systems Age having very different age-accelerated system scores. (C) Overrepresentation analysis of presence or absence of diseases amongst individuals from 9 different clusters. P Values have been calculated using fisher's exact test and are available in Table 10. (D) Mean age accelerated score has been depicted for each cluster using a spider plot and is also available in Table 11.

FIGS. 6A-K show graphs illustrating associations of biomarkers with system specific scores. (A) Blood. (B) Brain. (C) Heart. (D) Hormone. (E) Inflammation. (F) Immune. (G) Liver. (H) Kidney. (I) Lung. (J) Metabolic. (K) MusculoSkeletal. We used linear regression to model the association between the system scores with each biomarker in the Health and Retirement Study, reporting the Z-scores of association.

FIG. 7 shows graphs illustrating ranks of clocks based on z-scores with no adjustments, smoking status adjusted, and only non-smokers.

FIG. 8 shows a graph illustrating reliability of different age accelerated system scores and Systems Age as compared to other clocks (n=36).

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION

Provided herein are methods of training an algorithm to determine systems-level epigenetic scores. In some embodiments, the method includes grouping biomarkers into specific biological systems, deconvoluting the biological systems into principal components (PCs), building predictors of system PCs, and building system scores. The system scores represent mortality prediction scores, which are used as measures of aging and deterioration of a specific biological system. In some embodiments, the steps of grouping biomarkers, deconvoluting, and building predictors of system PCs utilize a first dataset including biomarker data and DNA methylation data. Additionally or alternatively, in some embodiments, the step of building system scores utilizes a second dataset including DNA methylation data and linked mortality data. Although described herein primarily with respect to two datasets, as will be appreciated by those skilled in the art, the disclosure is not so limited and may include any other suitable number of datasets.

The biomarkers include any suitable clinically relevant biomarker, such as, but not limited to, clinical chemistry biomarkers, functional biomarkers, system specific diseases, and/or system specific condition history. For example, in one embodiments, the biomarkers include clinical chemistry assays measured in plasma and serum, physiological measurements, functional tests, and history of symptoms and diseases. The biomarkers may also be grouped into any suitable number of different biological systems, such as, but not limited to, at least 9 systems, at least 10 systems, at least 11 systems, up to 11 systems, or any combination, sub-combination, range, or sub-range thereof. In one embodiment, for example, the biomarkers are grouped into the biological systems including blood, brain, cardiac, hormone, immune, inflammation, kidney, liver, lung, metabolic, and/or musculoskeletal. In another embodiment, the biomarkers are grouped into the systems as shown in Table 1.

TABLE 1
Example grouping of biomarkers into biological systems.

| System | Biomarkers and Phenotypes |
|---|---|
| Blood | Ferritin, Hematocrit, Hemoglobin, Mean Corpuscular |
| | Hemoglobin, Mean Corpuscular Hemoglobin Concentration, |
| | Mean Corpuscular Volume, Mean Platelet Volume, Platelet |
| | Distribution Width, Platelet Count, Red Blood Cell Count, |
| | Red Cell Distribution Width |
| Brain | Homocysteine, Serum BDNF, Clusterin, total mental status |
| | summary score, total cognition summary score, immediate |
| | word recall score, delayed word recall score, total word recall |
| | summary score, serial 7s test score, history of stroke |
| Cardiac | Homocysteine, BMI, systolic blood pressure, diastolic blood |
| | pressure, waist circumference, pulse, history of shortness of |
| | breath while awake, PC components of GrimAge |
| Hormone | Dehydroepiandrosterone sulphate, IGF1 |
| Immune | Eosinophil Count, Lymphocyte Count, Monocyte Count, |
| | Neutrophil Count, Basophils %, Eosinophils %, Lymphocytes |
| | %, Monocytes %, White Blood Cell Count, Myeloid |
| | Dendritic cells (DC-M) %, Plasmacytoid Dendritic Cells |
| | (DC-P) %, NK Cells: CD56HI %, NK Cells: CD56LO %, |
| | CD16− Monocytes %, CD16+ Monocytes %, B Cells %, |
| | CD8+ T Cells: Central Memory (CM) %, CD4+ T Cells: |
| | Central Memory (CM) %, CD8+ T Cells %, CD8+ T Cells: |
| | (TemRA) %, CD4+ T Cells: (TemRA) %, CD4+ T Cells %, |
| | IgD+ Memory B Cells %, IgD− Memory B Cells %, CD8+ T |
| | Cells: Naive %, CD4+ T Cells: Naive %, T Cells %, Naive B |
| | Cells %, CD8+ T Cells: Effector Memory (Tem) %, CD4+ T |
| | Cells: Effector Memory (Tem) %, NK Cells %, Monocytes |
| | %, Dendritic Cells % |
| Inflammation | C-Reactive Protein, Transforming Growth Factor Beta, |
| | Interleukin 10, Interleukin 1 Receptor Antagonist, Interleukin |
| | 6, Tumor Necrosis Factor Receptor 1, Ferritin |
| Kidney | Albumin, Urea Nitrogen, Chloride, Bicarbonate, Creatinine, |
| | Cystatin C, Potassium, Sodium |
| Liver | Albumin, Alkaline Phosphatase, ALT, AST, Bilirubin, Total |
| | Protein |
| Lung | Bicarbonate, PC prediction of smoking pack-years, history of |
| | chronic lung disease, history of shortness of breath while |
| | awake, history persistent wheezing/cough/phlegm, peak |
| | expiratory flow, receiving oxygen |
| Metabolic | C-Reactive Protein, Glucose-Fasting, HDL-Cholesterol, LDL- |
| | Cholesterol, Triglycerides, Interleukin-6, history of Diabetes, |
| | BMI, Waist circumference |
| Musculoskeletal | Vitamin D3, DHEASE, IGF1, history of arthritis, height, |
| | weight, BMI, history of difficulty with mobility, history of |
| | back problems, maximum grip strength, grip strength left and |
| | right, semi tandem balance test time, full tandem balance test |
| | time, side-by-side balance test time, timed walk test time, |
| | timed walk test time with walking aid, difficulty doing daily |
| | physical movements (stooping/kneeling/crouching, walking |
| | one block, walking several blocks, climbing several flights of |
| | stairs, climbing one flight of stairs, getting up from a chair, |
| | raising arms above one's head, carrying 10 lbs, picking up a |
| | dime) |

After grouping the biomarkers, the deconvolution step includes performing principal component analysis (PCA) on the biomarkers. In some embodiments, this step also includes separately performing PCA on DNA methylation data from the dataset. The performing of the PCA on the biomarkers and/or DNA methylation data generates a set of biomarker and/or methylation principal component (PC) scores for each individual in the dataset. This unsupervised learning step also provides an eigenvector matrix for deconvoluting the data. In some embodiments, the eigenvector matrix reduces the dimensionality of the data and removes collinearity, while retaining at least a majority of the relevant variation in the original data. Without wishing to be bound by theory, it is believed that de-convoluting the biomarkers into their specific components allows more subtle variations to be predicted as well as the more dominant components, thus giving them a chance to become part of the eventual prediction model.

The step of building predictors of system PCs includes applying an elastic net regression to the methylation PC scores to generate system PC predictors for each of the biological systems. In some embodiments, this step includes generating a model including the system PC predictors. In some embodiments, the elastic net regression includes a supervised elastic net penalized regression. In some embodiments, the L1 to L2 regularization ratio is kept at 1 or, in other words, the alpha parameter of the elastic net model is 0.5. In some embodiments, only some of the system PCs predicted using methylation PCs are retained. For example, in one embodiment, the method includes retains DNAm system PCs with at least 20 DNAm PCs being used at the minimum mean cross-validated error in the model and at least 5 DNAm PCs at the cross-validated error one standard error from the minimum mean cross-validated error in the model. In some embodiments, this provides only well predicted DNAm system PCs to the next step.

The step of building system scores includes combining the DNAm system PCs in a cox elastic net mortality prediction model in R. In some embodiments, this includes recalculating the DNAm system PCs based on parameters previously trained the step of building predictors of system PCs. For example, in one embodiment, this step includes applying the eigenvector matrices to a second dataset including DNA methylation data and linked mortality data, which generates estimated DNA methylation PC scores for each individual in the second dataset. These estimated DNA methylation PC scores are then input into the model including the system PC predictors to produce DNA methylation proxies for system-specific PC scores. Finally, the DNA methylation proxies of system-specific PC scores are separately input into a Cox elastic net penalized regression for each of the biological system to generate mortality prediction scores for each of the biological systems. These mortality prediction scores are used as measures of aging and deterioration of a system, and form system-specific epigenetic age predictor for each of the biological systems, also referred to herein as system scores.

Additionally or alternatively, in some embodiments, the method includes training the algorithm to predict a combined systems age score. In some embodiments, predicting the combined systems age score includes performing Cox elastic net penalized regression to predict mortality using a combination of the system-specific epigenetic age predictor for each of the biological systems to generate a combined systems age measure. In some embodiments, generating the combined systems age measure reduces redundancy and allows for smaller variations. Furthermore, in some embodiments, the combined systems age measure predicts aging phenotypes without a bias towards particular phenotypes.

Also provided herein, in some embodiments, are methods of calculating systems-level epigenetic scores. The method includes applying the algorithm according to any of the embodiments disclosed herein to a blood sample from a subject, and calculating epigenetic scores for individual biological systems based upon data derived from the blood sample using the algorithm. In some embodiments, the method further includes calculating a combined systems age measure using the algorithm according to one or more of the embodiments disclosed herein.

Further provided herein are an apparatus for calculating systems-level epigenetic scores and a computer readable storage medium storing computer-executable instructions for performing the method according to any of the embodiments disclosed herein. In some embodiments, the apparatus includes a processor, a memory unit, and a communication interface. The processor is connected to the memory unit and the communication interface, and the processor and memory are configured to implement the method.

As opposed to existing articles and methods, which calculate a single epigenetic age, the articles and methods disclosed herein provide estimation of multiple distinct epigenetic age measures that each map to specific physiological systems. This estimation of multiple distinct epigenetic age measures provides more detailed information that is relevant to patient health, resulting in distinct profiles not found using existing epigenetic clocks. Additionally or alternatively, because the systems age measurement disclosed herein incorporates multiple systems, it predicts all examined aging outcomes well, while previously reported epigenetic clocks predict some outcomes well but not others. Furthermore, by predicting system-level scores, the articles and methods disclosed herein provide information about which specific age-related diseases or types of functional decline a person is at risk for. In contrast, a traditional epigenetic clock would only indicate if a person is at higher risk for diseases of aging in general. This clinical interpretability and specificity of systems-specific epigenetic clocks may be further applied to clinical prevention, screening, diagnosis, prognosis, and treatment of specific age-related diseases.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

Individuals, organs, tissues, and cell types age in diverse ways throughout the lifespan. Epigenetic clocks attempt to quantify differential aging between individuals, but they typically summarize aging as a single measure, ignoring within-person heterogeneity. This Example describes the development of systems-based methylation clocks that, when assessed in blood, captured aging in distinct physiological systems.

The overarching aim of this study was to construct systems-specific aging scores from DNA methylation data derived from whole blood. While clinical biomarkers and functional measures themselves provide direct assessment of specific physiological systems, we reasoned systems-specific methylation predictors would have two major advantages: 1) it is a single standardized assay that is comparable between studies, whereas the set of clinical biomarkers and functional measures can markedly differ between aging studies, and 2) DNA methylation is closer to root causes of aging (López-Otín, C., Blasco, M. A., Partridge, L., Serrano, M. & Kroemer, G. The hallmarks of aging. Cell 153, 1194-1194 (2013)). We found it is possible to capture heterogeneity across many physiological systems using a single blood DNA methylation test, in turn predicting decline and disease specific to each system as well as cluster individuals into diverse yet distinct epigenetic aging subtypes.

Supervised and unsupervised machine learning methods were combined to link DNA methylation, system-specific clinical chemistry and functional measures, and mortality risk. This yielded a panel of 11 system-specific scores-Heart, Lung, Kidney, Liver, Brain, Immune, Inflammatory, Blood, Musculoskeletal, Hormone, and Metabolic. Each system score predicted a wide variety of outcomes, aging phenotypes and conditions specific to each system, and often did so more strongly than existing epigenetic clocks that report single global measures. The systems scores were also combined into a composite Systems Age clock that is predictive of aging across physiological systems in an unbiased manner. Finally, it is shown that the system scores clustered individuals into unique aging subtypes that had different patterns of age-related disease and decline. Overall, the biological systems based epigenetic framework captures aging in multiple physiological systems using a single blood draw, which can be applied to more personalized clinical approaches for improving age-related quality of life.

Not only can this refinement of aging indicators help predict disease-specific differential risks across individuals, the understanding of heterogeneity in aging may facilitate targeted interventions based on personalized aging characteristics.

### Results

### Systems Age Pipeline for Modeling Systems-Specific Aging

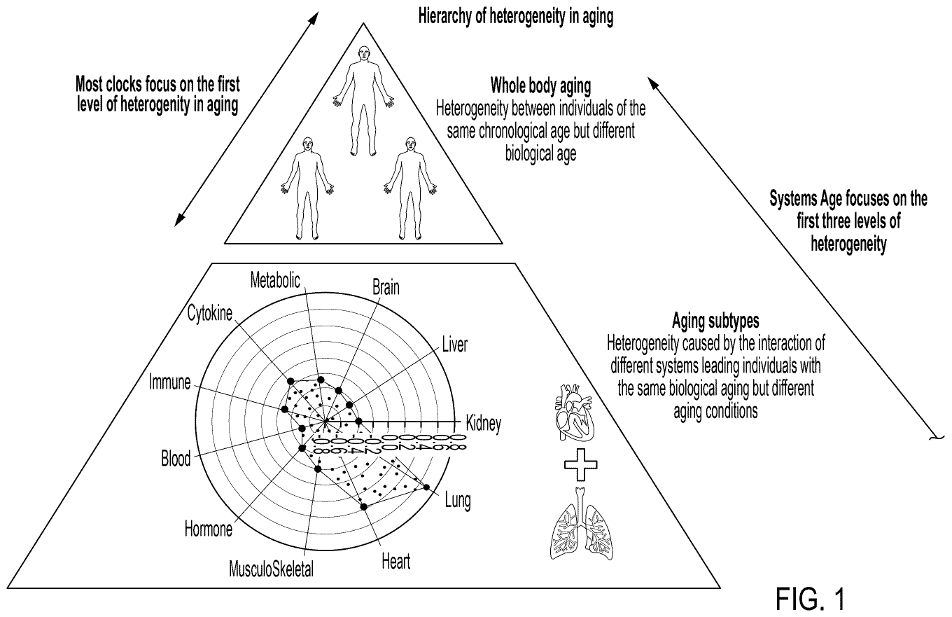

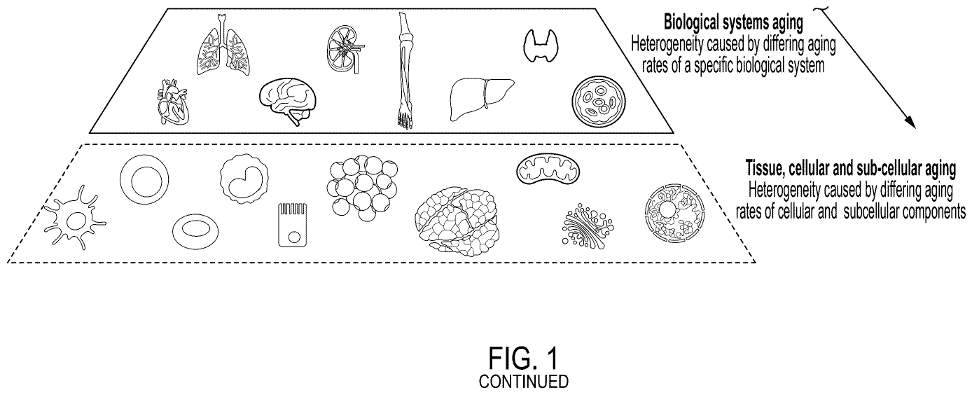

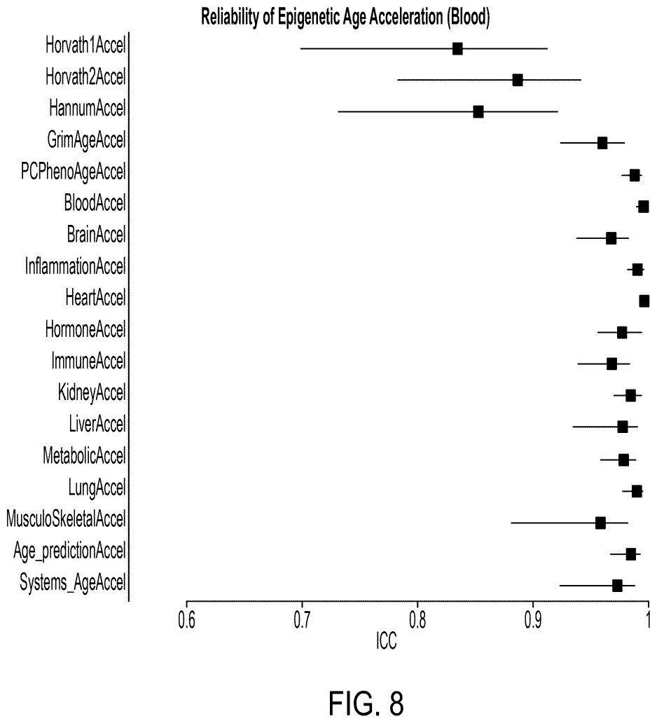

Systems Age was constructed in a five-step process (FIG. 1, Methods). Briefly, we first mapped clinical chemistry biomarkers available in the Health and Retirement study (HRS) to specific biological systems (Table 2). In addition to blood-based measures, we incorporated relevant functional assessments and disease status. Second, we performed principal component analysis (PCA) on measures within each system to identify latent signals captured by system-specific principal components (PCs). Third, we predicted these system PCs using methylation PCs selected via elastic net regression within HRS. We utilized methylation PCs for predicting these measures given that use of PCs over individual CpGs increases test-retest reliability without sacrificing validity (Higgins-Chen, A. T., Thrush, K. L., Wang, Y. & Minteer, C. J. A computational solution for bolstering reliability of epigenetic clocks: Implications for clinical trials and longitudinal tracking. (2022)) (FIG. 8). Fourth, we calculated the predicted DNAm system PCs (using the models trained in HRS) in the Framingham heart Study (FHS) and trained a mortality prediction model for each system via elastic net Cox penalized regression. We referred to the resulting scores as ‘system scores’, meant to estimate aging in a particular system. Finally, we trained an elastic net Cox model by incorporating PCs from all systems into a unified whole-body score called ‘Systems Age’. Both individual system scores and overall System Age were scaled to the expected age range for interpretability.

TABLE 2
Biomarker to systems mapping and biomarker transformation

| | | Dataset-Specific | | |
|---|---|---|---|---|
| Dataset | System | Variable Name | Transformation | Biomarker |
| HRS | Blood | PFERTN | PFERTN**(1/1.75 | Ferritin |
| HRS | Blood | PHCT | PHCT | Hematocrit |
| HRS | Blood | PHGB | PHGB**1.18 | Hemoglobin |
| HRS | Blood | PMCH | PMCH\hat{ }1.4 | Mean Corpuscular |
| | | | | Hemoglobin |
| HRS | Blood | PMCHC | PMCHC\hat{ }1.2 | Mean Corpuscular |
| | | | | Hemoglobin Conc |
| HRS | Blood | PMCV | PMCV\hat{ }1.2 | Mean Corpuscular Volume |
| HRS | Blood | PMPV | PMPV\hat{ }(1/1.3) | Mean Platelet Volume |
| HRS | Blood | PPDW | PPDW\hat{ }(1/1.75) | Platelet Distribution width |
| HRS | Blood | PPLT | PPLT\hat{ }(1/1.3) | Platelet Count |
| HRS | Blood | PRBC | PRBC | Red Blood Cell Count |
| HRS | Blood | PRDW | PRDW\hat{ }(1/2) | Red Cell Distribution Width |
| HRS | Brain | phcy | phcy\hat{ }(1/1.9) | Homocysteine |
| HRS | Brain | pbdnf | pbdnf\hat{ }(1/1.1) | BDNF (serum) |
| HRS | Brain | pclusterin | pclusterin\hat{ }(1/1.28) | clusterin |
| HRS | Brain | r13stroke | r13stroke | Stroke |
| HRS | Brain | r13mstot | r13mstot | total mental status summary |
| | | | | score |
| HRS | Brain | r13cogtot | r13cogtot | total cognition summary |
| | | | | score |
| HRS | Brain | r13imrc | r13imrc | immediate word recall |
| HRS | Brain | r13dlrc | r13dlrc | delayed word recall |
| HRS | Brain | r13tr20 | r13tr20 | total word recall summary |
| | | | | score |
| HRS | Brain | r13ser7 | r13ser7 | serial 7s |
| HRS | Cardiac | PC144 | PC144 | Shortness of breath while |
| | | | | awake |
| HRS | Cardiac | — | — | PCcomponents of Grimage |
| HRS | Cardiac | r13pmwaist | r13pmwaist | Waist circumference |
| HRS | Cardiac | r13bppuls | r13bppuls | Pulse |
| HRS | Cardiac | r13bpdia, r13bpsys | r13bpdia, r13bpsys | BP |
| HRS | Cardiac | phcy | phcy\hat{ }(1/1.9) | Homocysteine |
| HRS | Cardiac | r13bmi | r13bmi | BMI |
| HRS | Hormone | PIGF1 | PIGF1\hat{ }(1/1.4) | IGF1 |
| HRS | Hormone | PDHEASE | PDHEASE)\hat{ }(1/1.4) | DHEAS |
| HRS | Inflammation | PFERTN | PFERTN\hat{ }(1/3) | Ferritin |
| HRS | Inflammation | PCRP | PCRP\hat{ }(1/2.8) | C-Reactive Protein |
| HRS | Inflammation | PTGF_BETA | PTGF_BETA\hat{ }(1/1.35) | Transforming Growth |
| | | | | Factor Beta |
| HRS | Inflammation | PIL10 | PIL10\hat{ }(1/2.05) | Interleukin 10 |
| HRS | Inflammation | PIL1RA | PIL1RA\hat{ }(1/2) | Interleukin 1 Receptor |
| | | | | Antagonist |
| HRS | Inflammation | PIL6 | PIL6\hat{ }(1/2.95) | Interleukin 6 |
| HRS | Inflammation | PTNFR1 | PTNFR1\hat{ }(1/2.05) | Tumor Necrosis Factor |
| | | | | Receptor 1 |
| HRS | Immune | PAEOS_resid_fast | PAEOS_resid_fast | Eosinophil Count |
| HRS | Immune | PALYM_resid_fast | PALYM_resid_fast\hat{ }(1/1.65) | Lymphocyte Count |
| HRS | Immune | PAMON_resid_fast | PAMON_resid_fast\hat{ }(1/1.65) | Monocyte Count |
| HRS | Immune | PANEU_resid_fast | PANEU_resid_fast\hat{ }(1/1.65) | Neutrophil Count |
| HRS | Immune | PBASO_resid_fast | PBASO_resid_fast | Percent Basophils |
| HRS | Immune | PEOS_resid_fast | PEOS_resid_fast\hat{ }(1/1.2) | Percent Eosinophils |
| HRS | Immune | PLYMP_resid_fast | PLYMP_resid_fast\hat{ }(1/1.27) | Percent Lymphocytes |
| HRS | Immune | PMONO_resid_fast\hat{ }(1/1.45) | PMONO_resid_fast\hat{ }(1/1.45) | Percent Monocytes |
| HRS | Immune | PWBC_resid_fast\hat{ }(1/1.6) | PWBC_resid_fast\hat{ }(1/1.6) | White Blood Cell Count |
| HRS | Immune | PDCM_PCT | PDCM_PCT\hat{ }(2.3) | Myeloid Dendritic cells (DC- |
| | | | | M) Percentage |
| HRS | Immune | PDCP_PCT | PDCP_PCT\hat{ }(1/2.9) | Plasmacytoid Dendritic Cells |
| | | | | (DC-P) Percentage |
| HRS | Immune | PNKHI_PCT | PNKHI_PCT\hat{ }(1/7.1) | NK Cells: CD56HI |
| | | | | Percentage |
| HRS | Immune | PNKLO_PCT | PNKLO_PCT\hat{ }(12) | NK Cells: CD56LO |
| | | | | Percentage |
| HRS | Immune | PMONO16MINUS_PCT | PMONO16MINUS_PCT\hat{ }(12) | CD16− Monocytes |
| | | | | Percentage |
| HRS | Immune | PMONO16PLUS_PCT | PMONO16PLUS_PCT\hat{ }(1/5.25) | CD16+ Monocytes |
| | | | | Percentage |
| HRS | Immune | PBCELL_PCT | PBCELL_PCT\hat{ }(1/4.1) | B Cells Percentage |
| HRS | Immune | PCD8M_PCT | PCD8M_PCT\hat{ }(1/3.55) | CD8+ T Cells: Central |
| | | | | Memory (CM) Percentage |
| HRS | Immune | PCD4M_PCT | PCD4M_PCT\hat{ }(1/1.2) | CD4+ T Cells: Central |
| | | | | Memory (CM) Percentage |
| HRS | Immune | PCD8T_PCT | PCD8T_PCT\hat{ }(1/2.7) | CD8+ T Cells Percentage |
| HRS | Immune | PCD8TEMRA_PCT | PCD8TEMRA_PCT\hat{ }(1/1.2) | CD8+ T Cells: (TemRA) |
| | | | | Percentage |
| HRS | Immune | PCD4TEMRA_PCT | PCD4TEMRA_PCT\hat{ }(1/6) | CD4+ T Cells: (TemRA) |
| | | | | Percentage |
| HRS | Immune | PCD4T_PCT | PCD4T_PCT\hat{ }(2) | CD4+ T Cells Percentage |
| HRS | Immune | PIGD_PLUS_MEMB_PCT | PIGD_PLUS_MEMB_PCT\hat{ }(1/3.3) | IgD+ Memory B Cells |
| | | | | Percentage |
| HRS | Immune | PIGD_MINUS_MEMB_PCT | PIGD_MINUS_MEMB_PCT\hat{ }(1/3.5) | IgD− Memory B Cells |
| | | | | Percentage |
| HRS | Immune | PCD8N_PCT | PCD8N_PCT\hat{ }(1/2.4) | CD8+ T Cells: NaÔve |
| | | | | Percentage |
| HRS | Immune | PCD4N_PCT | PCD4N_PCT\hat{ }(1/1.2) | CD4+ T Cells: NaÔve |
| | | | | Percentage |
| HRS | Immune | PTCELL_PCT | PTCELL_PCT\hat{ }(2.25) | T Cells Percentage |
| HRS | Immune | PNAIVEB_PCT | PNAIVEB_PCT\hat{ }(2) | Naive B Cells Percentage |
| HRS | Immune | PCD8TEM_PCT | PCD8TEM_PCTA\hat{ }(1/7.1) | CD8+ T Cells: Effector |
| | | | | Memory (Tem) Percentage |
| HRS | Immune | PCD4TEM_PCT | PCD4TEM_PCTA\hat{ }(1/7) | CD4+ T Cells: Effector |
| | | | | Memory (Tem) Percentage |
| HRS | Immune | PNK_PCT | PNK_PCT\hat{ }(1/5.1) | Natural Killer (NK) Cells |
| | | | | Percentage |
| HRS | Immune | PMONO_PCT | PMONO_PCT\hat{ }(1/4.6) | Monocytes Percentage |
| HRS | Immune | PDC_PCT | PDC_PCT\hat{ }(1/7.5) | Dendritic Cells Percentage |
| HRS | Kidney | PALB_resid_fast | PALB_resid_fast\hat{ }(1.2) | Albumin |
| HRS | Kidney | PBUN_resid_fast | PBUN_resid_fast\hat{ }(1/1.4) | Urea Nitrogen |
| HRS | Kidney | PCL_resid_fast | PCL_resid_fast\hat{ }(1.5) | Chloride |
| HRS | Kidney | PCO2_resid_fast | PCO2_resid_fast | Bicarbonate |
| HRS | Kidney | PCR_resid_fast | PCR_resid_fast\hat{ }(1/2.35) | Creatinine |
| HRS | Kidney | PCYSC_resid_fast | PCYSC_resid_fast\hat{ }(1/2.15) | Cystatin C |
| HRS | Kidney | PK_resid_fast | PK_resid_fast\hat{ }(1/1.2) | Potassium |
| HRS | Kidney | PNA_resid_fast | PNA_resid_fast\hat{ }(1.25) | Sodium |
| HRS | Liver | PALB_resid_fast | PALB_resid_fast\hat{ }(1.2) | Albumin |
| HRS | Liver | PALKP2_resid_fast | PALKP2_resid_fast\hat{ }(1/1.8) | Alkaline Phosphatase |
| HRS | Liver | PALT_resid_fast | PAST_resid_fast\hat{ }(1/2.1) | ALT |
| HRS | Liver | PAST_resid_fast | PAST_resid_fast\hat{ }(1/2.1) | AST |
| HRS | Liver | PBILT_resid_fast | PBILT_resid_fast\hat{ }(1/1.55) | Bilirubin |
| HRS | Liver | PTP_resid_fast | PTP_resid_fast\hat{ }(1/1.11) | Total Protein |
| HRS | Lung | puff | puff | Peak expiratory flow |
| HRS | Lung | PCO2 | PCO2 | Bicarbonate |
| HRS | Lung | PC030 | PC030 | Chronic lung disease |
| HRS | Lung | PC033 | PC033 | Receiving oxygen |
| HRS | Lung | PC144 | PC144 | Shortness of breath while |
| | | | | awake |
| HRS | Lung | PC149 | PC149 | Persistent wheezing, cough, |
| | | | | or bringing up phlegm |
| HRS | Lung | PC-packyears | PC-packyears | PCSmoking-packyears |
| HRS | Metabolic | PCRP_resid_fast | PCRP_resid_fast\hat{ }(1/4.5) | C-Reactive Protein |
| HRS | Metabolic | PGLUFF_resid_fast | PGLUFF_resid_fast\hat{ }(1/2.6) | Glucose-Fasting |
| HRS | Metabolic | PHDLD_resid_fast | PHDLD_resid_fast\hat{ }(1/1.8) | HDL-Cholesterol |
| HRS | Metabolic | PLDLC_resid_fast | PLDLC_resid_fast\hat{ }(1/1.35 | LDL-Cholesterol |
| HRS | Metabolic | PTGF_resid_fast | PTGF_resid_fast\hat{ }(1/2.1) | Triglycerides |
| HRS | Metabolic | PIL6_resid_fast | PIL6_resid_fast\hat{ }(1/4.6) | Interleukin-6 |
| HRS | Metabolic | r13bmi | r13bmi | BMI |
| HRS | Metabolic | r13pmwaist | r13pmwaist | Waist circumference |
| HRS | Musculoskeletal | PVD3 | PVD3_resid_fast\hat{ }(1/3) | Vitamin D3 |
| HRS | Musculoskeletal | PIGF1 | PIGF1\hat{ }(1/1.4) | Dehydroepiandrosterone |
| | | | | sulphate |
| HRS | Musculoskeletal | PDHEASE | PDHEASE)\hat{ }(1/1.4) | IGF1 |
| HRS | Musculoskeletal | r13arthre | r13arthre | Arthritis |
| HRS | Musculoskeletal | r13height | r13height | Height |
| HRS | Musculoskeletal | r13weight | r13weight | Weight |
| HRS | Musculoskeletal | r13bmi | r13bmi | BMI |
| HRS | Musculoskeletal | r13mobila | r13mobila | some diff-mobility |
| HRS | Musculoskeletal | r13grp | r13grp | hand grip strength maximum |
| | | | | measurement |
| HRS | Musculoskeletal | r13balsemi | r13balsemi | semi tandem balance test |
| | | | | time |
| HRS | Musculoskeletal | r13timwlk | r13timwlk | timed walk test time |
| HRS | Musculoskeletal | r13timwlka | r13timwlka | timed walk test-walking aid |
| | | | | used |
| HRS | Musculoskeletal | r13grpl | r13grpl\hat{ }(1/2.65) | hand grip strength-left hand |
| HRS | Musculoskeletal | r13grpr | r13grpr\hat{ }(1/2.65) | hand grip strength-right hand |
| HRS | Musculoskeletal | r13back | r13back | had back problems |
| HRS | Musculoskeletal | r13stoop | r13stoop | r diff-stoop/kneel/crouch |
| HRS | Musculoskeletal | r13walk1 | r13walk1 | diff-walk one block |
| HRS | Musculoskeletal | r13walks | r13walks | diff-walk sev blocks |
| HRS | Musculoskeletal | r13walk1a | r13walk1a | some diff-walk one block |
| HRS | Musculoskeletal | r13walksa | r13walksa | some diff-walk sev blocks |
| HRS | Musculoskeletal | r13clims | r13clims | diff-climb sev flt stair |
| HRS | Musculoskeletal | r13clim1 | r13clim1 | diff-climb one flt stair |
| HRS | Musculoskeletal | r13climsa | r13climsa | some diff-clmb sev flt str |
| HRS | Musculoskeletal | r13clim1a | r13clim1a | some diff-clmb 1 flt stair |
| HRS | Musculoskeletal | r13chair | r13chair | diff-get up fr chair |
| HRS | Musculoskeletal | r13chaira | r13chaira | some diff-get up fr chair |
| HRS | Musculoskeletal | r13arms | r13arms | diff-reach/extnd arms up |
| HRS | Musculoskeletal | r13armsa | r13armsa | some diff-rch/xtnd arms up |
| HRS | Musculoskeletal | r13lift | r13lift | diff-lift/carry 10lbs |
| HRS | Musculoskeletal | r13lifta | r13lifta | some diff-lift/carry 10lbs |
| HRS | Musculoskeletal | r13dime | r13dime | diff-pick up a dime |
| HRS | Musculoskeletal | r13dimea | r13dimea | some diff-pick up a dime |
| HRS | Musculoskeletal | r13balsbs | r13balsbs | side-by-side balance test time |
| HRS | Musculoskeletal | r13balful | r13balful | full tandem balance test time |

### System Scores Capture Meaningful and Specific Aging Signals

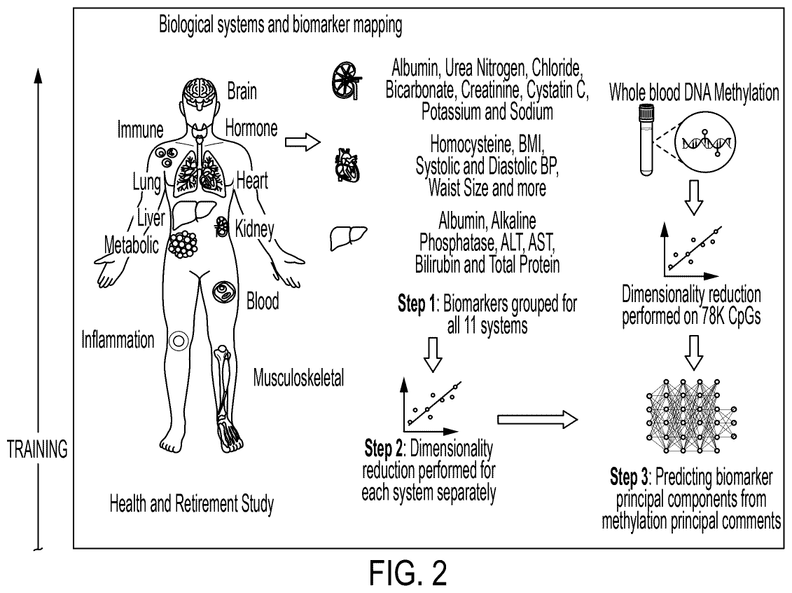

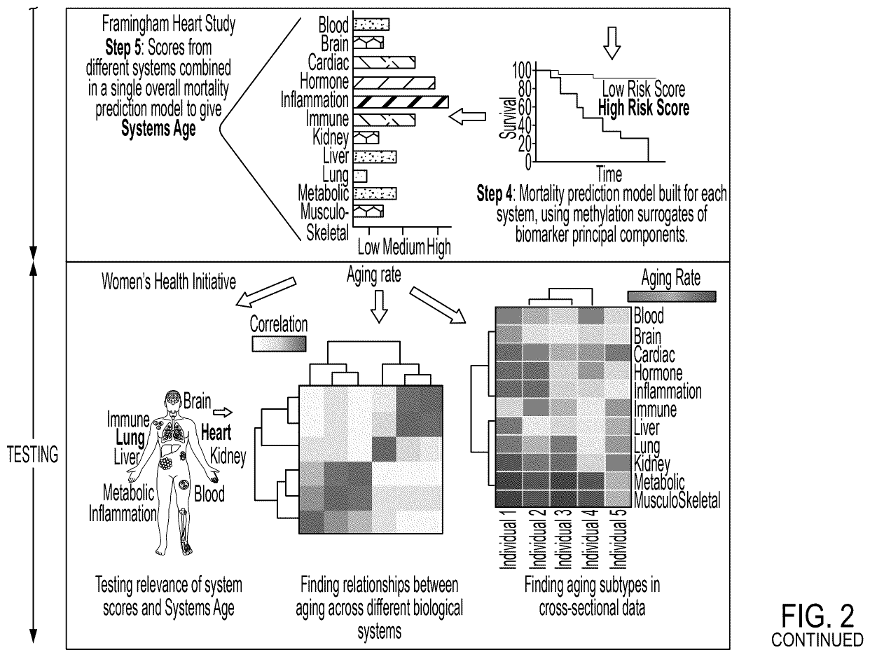

Specificity of system scores was assessed in an independent sample from 3 cohorts of the Women's Health Initiative (WHI BAA23, AS311 and EMPC, total N=~5,600). Details about each cohort are available in the Methods section. Cohorts were stratified by race (except WHI AS311 which included few black and hispanic participants), for a total of 7 groups. Results from multivariate analyses adjusting for chronological age were meta-analyzed via a fixed effects model with inverse variance weights (FIG. 2 as well as Tables 3 and 4). We tested associations with disease incidence (using cox proportional hazard models), disease prevalence (using logistic regression models), and functional parameters of aging (using Ordinary Least Squares regression models).

TABLE 3
Z-scores for meta-analysis (adjusted for age)

| | Blood | Brain | Inflammation | Heart | Hormone | Immune |
|---|---|---|---|---|---|---|
| Cognitive | 2.926154646 | 3.513373784 | 3.106784239 | 2.559157421 | 1.693460401 | 2.410753019 |
| function | | | | | | |
| Physical | 5.32615051 | 6.178750508 | 8.532305851 | 8.838268962 | 5.271089588 | 3.925931716 |
| function | | | | | | |
| CHD | 2.893738483 | 3.467296151 | 6.830712916 | 8.299655288 | 4.533141311 | 5.324501954 |
| MI | 1.661292032 | 1.972331812 | 4.879202323 | 6.302134584 | 3.258920503 | 4.417790861 |
| Any Cancer | −1.412736401 | −0.581055182 | 0.758667594 | 4.372246526 | 0.100961174 | 1.346579621 |
| Lung Cancer | 1.362149928 | 1.915070114 | 3.718141971 | 9.494544155 | 1.223192471 | 3.352558031 |
| Breast Cancer | −2.561570498 | −1.160978572 | −1.147100709 | −0.989650959 | −3.043317512 | −1.265912667 |
| Stroke | 1.732413133 | 2.804747999 | 3.063212162 | 3.404102966 | 1.330674767 | 2.417934984 |
| Mortality | 6.337331526 | 6.47791523 | 11.47688711 | 15.17566945 | 6.685692775 | 7.168520717 |
| Total | 3.084165133 | 4.626134858 | 7.65137464 | 7.452231469 | 4.148956456 | 3.961856206 |
| comorbidities | | | | | | |
| Disease free | 2.356734717 | 3.000284853 | 2.410542486 | 3.986868062 | 2.233630703 | 1.733394003 |
| Diabetes | 3.558469784 | 4.000451402 | 8.119187225 | 6.074247748 | 4.870005129 | 1.242043086 |
| Colorectal | 2.085160502 | 0.730460164 | 0.638663969 | 0.5819431 | 1.489498965 | 0.458220821 |
| cancer | | | | | | |
| Endometrial | −2.220059298 | −1.791910189 | −1.726864327 | −1.552967708 | −1.277998404 | −0.354012827 |
| cancer | | | | | | |
| Leukemia | 4.886218892 | 3.757749624 | 3.092247777 | 2.432887385 | 2.916228189 | 3.697671088 |
| Arthritis | 1.488053827 | 1.929599749 | 4.430245903 | 3.288359763 | 1.99413665 | 0.782802202 |
| Thyroid | 0.07302894 | 0.016423901 | 1.172636506 | 2.434849738 | 2.656835559 | 0.690136329 |
| disease | | | | | | |
| Cataract | 2.868483532 | 2.953921401 | 2.147152699 | 3.166815541 | 1.908864697 | 1.104290093 |

| | Kidney | Liver | Metabolic | Lung | MusculoSkeletal |
|---|---|---|---|---|---|
| Cognitive | 2.962496239 | 2.2565478 | 2.440880814 | 1.983392772 | 1.045837891 |
| function | | | | | |
| Physical | 6.431810249 | 5.540548142 | 7.699911489 | 5.603359314 | 9.46038496 |
| function | | | | | |
| CHD | 5.307156251 | 3.586320438 | 5.658107319 | 6.452076336 | 5.364644837 |
| MI | 3.743128142 | 2.529642619 | 3.76304316 | 5.000222616 | 3.256403604 |
| Any Cancer | −0.014837834 | −0.698371012 | −0.333840321 | 5.792538494 | 0.384726405 |
| Lung Cancer | 2.235756218 | 1.488690537 | 2.393432246 | 9.696366397 | 0.740260148 |
| Breast Cancer | −1.637198326 | −1.050524852 | −2.025457655 | −0.945730374 | −0.231505216 |
| Stroke | 1.494041698 | 1.93304693 | 3.352701129 | 1.767708053 | 2.284356707 |
| Mortality | 8.808971824 | 5.83633678 | 9.5574104 | 12.89051239 | 8.028807498 |
| Total | 5.780661337 | 4.372632718 | 6.498500802 | 4.060700008 | 7.594057379 |
| comorbidities | | | | | |
| Disease free | 2.283243524 | 2.893960868 | 2.671556564 | 2.601626563 | 2.417828491 |
| Diabetes | 4.983233328 | 3.865640567 | 8.260480933 | 2.968395133 | 10.90448007 |
| Colorectal | 1.238428126 | 1.351645017 | 1.201119376 | 0.292874568 | 1.347619374 |
| cancer | | | | | |
| Endometrial | −0.95981025 | −0.801248093 | −2.096955376 | −0.92917327 | −1.284541733 |
| cancer | | | | | |
| Leukemia | 4.156339387 | 2.276391683 | 3.22759013 | 3.214400417 | 2.283183273 |
| Arthritis | 3.49982054 | 2.252437114 | 3.008568434 | 1.271076107 | 5.155457224 |
| Thyroid | 0.513659581 | −0.212583041 | 0.353594161 | 1.99996669 | 0.852823947 |
| disease | | | | | |
| Cataract | 2.222840161 | 3.120711648 | 2.897245256 | 1.541491447 | 1.623297921 |

| | Age_prediction | Systems_Clock | DunedinPACE | DNAmGrimAge | PCPhenoAge |
|---|---|---|---|---|---|
| Cognitive | 1.498970737 | 2.649655178 | 1.866942808 | 1.851945048 | 2.958494565 |
| function | | | | | |
| Physical | 5.353653133 | 9.099306917 | 9.477561719 | 7.080058529 | 8.357503484 |
| function | | | | | |
| CHD | 2.468443477 | 8.283725136 | 6.998012572 | 8.117840469 | 5.845892864 |
| MI | 0.648575243 | 6.178373515 | 5.606624994 | 6.099911092 | 3.441185951 |
| Any Cancer | 0.243896419 | 4.181854771 | 3.005942726 | 7.743231733 | 1.287245719 |
| Lung Cancer | 0.98763232 | 9.134368 | 4.936591676 | 12.19362204 | 3.187905194 |
| Breast Cancer | −1.661335385 | −1.115656328 | 0.846284446 | −0.272042581 | −1.267608383 |
| Stroke | 0.480638096 | 3.321700363 | 3.45722773 | 2.759082457 | 2.360604018 |
| Mortality | 5.721617101 | 15.12435643 | 11.05573492 | 16.81629829 | 10.66775857 |
| Total | 4.184398318 | 7.556498549 | 9.089695578 | 6.482483159 | 7.539989688 |
| comorbidities | | | | | |
| Disease free | 2.786973349 | 3.979772984 | 4.281589202 | 3.863818763 | 3.221136013 |
| Diabetes | 2.704614568 | 6.440487515 | 8.963196141 | 6.25264515 | 7.262762065 |
| Colorectal | 1.888083015 | 0.720885406 | 1.010529889 | 1.801063564 | 1.302125406 |
| cancer | | | | | |
| Endometrial | −0.664011685 | −1.595560084 | −0.669733532 | 0.546956906 | −1.198778241 |
| cancer | | | | | |
| Leukemia | 3.715884514 | 2.849418663 | 0.824593621 | 0.533156149 | 2.747760991 |
| Arthritis | 1.542498166 | 3.39167637 | 4.960231451 | 1.761386771 | 3.037000238 |
| Thyroid | 1.517050147 | 2.344812159 | 1.593929175 | 2.929206228 | 1.748929712 |
| disease | | | | | |
| Cataract | 2.83733683 | 3.167208264 | 2.375836947 | 2.136784047 | 2.597862574 |

| | Horvath1 | Horvath2 | Hannum |
|---|---|---|---|
| Cognitive | 0.723721146 | 0.664463117 | 2.208314547 |
| function | | | |
| Physical | 3.029940241 | 3.97919279 | 3.868751571 |
| function | | | |
| CHD | 1.071035137 | 0.977555528 | 2.312635661 |
| MI | 0.336692209 | −0.43459959 | 1.224814498 |
| Any Cancer | 1.006185299 | −0.097879064 | −0.639253948 |
| Lung Cancer | 2.20803603 | 0.949830531 | 0.490197465 |
| Breast Cancer | −1.008239545 | −2.861613113 | −3.641177408 |
| Stroke | 0.239180993 | 0.419495439 | 2.478456488 |
| Mortality | 2.325310461 | 3.885027871 | 4.855108718 |
| Total | 2.636817409 | 2.955720052 | 3.370769557 |
| comorbidities | | | |
| Disease free | 3.501148751 | 1.108763857 | 1.790391694 |
| Diabetes | 2.232491768 | 1.753820559 | 2.255838731 |
| Colorectal | 0.261133337 | 1.037395921 | 1.666047914 |
| cancer | | | |
| Endometrial | 1.097828281 | −0.603447907 | 0.437563214 |
| cancer | | | |
| Leukemia | 1.803099388 | 2.894536524 | 2.747740394 |
| Arthritis | 0.701338334 | 0.358893988 | 0.031998053 |
| Thyroid | 1.893669781 | 1.267340883 | 1.809593 |
| disease | | | |
| Cataract | 0.255580799 | 1.62815834 | 1.661118036 |

TABLE 4
P-values for heterogeneity in meta-analysis (adjusted for age)

| | Blood | Brain | Inflammation | Heart | Hormone | Immune |
|---|---|---|---|---|---|---|
| Cognitive | 0.178753174 | 0.432788594 | 0.21532822 | 0.480917194 | 0.473937326 | 0.05046351 |
| function | | | | | | |
| Physical | 0.071591949 | 0.444657722 | 0.589000766 | 0.024151295 | 0.387132872 | 0.355784282 |
| function | | | | | | |
| CHD | 0.971311056 | 0.730713736 | 0.299157339 | 0.405799113 | 0.615365161 | 0.563233735 |
| MI | 0.825179747 | 0.466650338 | 0.307151877 | 0.510290409 | 0.789626997 | 0.320170012 |
| Any Cancer | 0.395802904 | 0.198128175 | 0.304176863 | 0.491480073 | 0.372524245 | 0.401530209 |
| Lung Cancer | 0.796970649 | 0.218551492 | 0.062051636 | 0.043505604 | 0.577494836 | 0.837226909 |
| Breast Cancer | 0.54047199 | 0.68893262 | 0.391651095 | 0.502029456 | 0.193310974 | 0.483527145 |
| Stroke | 0.219654843 | 0.362509169 | 0.781757453 | 0.765150246 | 0.162487214 | 0.1393773 |
| Mortality | 0.808135736 | 0.289108945 | 0.033598839 | 0.62390785 | 0.170539704 | 0.607898212 |
| Total | 0.384505219 | 0.52081748 | 0.251332564 | 0.717449636 | 0.285040501 | 0.43620433 |
| comorbidities | | | | | | |
| Disease free | 0.66337724 | 0.480701744 | 0.279459164 | 0.079494237 | 0.248155253 | 0.263984445 |
| Diabetes | 0.856165131 | 0.603128408 | 0.79822093 | 0.882751622 | 0.33365897 | 0.788290649 |
| Colorectal | 0.980358055 | 0.63816658 | 0.75161839 | 0.527015972 | 0.65798498 | 0.283002948 |
| cancer | | | | | | |
| Endometrial | 0.933263089 | 0.390856769 | 0.451608914 | 0.882980556 | 0.570937668 | 0.600682135 |
| cancer | | | | | | |
| Leukemia | 0.913046996 | 0.897804555 | 0.08657606 | 0.995835294 | 0.09328732 | 0.321838263 |
| Arthritis | 0.807252007 | 0.694430146 | 0.822193278 | 0.814054968 | 0.567799737 | 0.43907895 |
| Thyroid | 0.274927611 | 0.17499784 | 0.24371471 | 0.295313886 | 0.371011804 | 0.928634142 |
| disease | | | | | | |
| Cataract | 0.310138564 | 0.284060014 | 0.059941415 | 0.098128348 | 0.122712866 | 0.082829457 |

| | Kidney | Liver | Metabolic | Lung | MusculoSkeletal |
|---|---|---|---|---|---|
| Cognitive | 0.333180821 | 0.140950501 | 0.349760476 | 0.249447377 | 0.942689988 |
| function | | | | | |
| Physical | 0.416928716 | 0.615470199 | 0.080985468 | 0.580661165 | 0.187312387 |
| function | | | | | |
| CHD | 0.904651069 | 0.735434414 | 0.559818869 | 0.620060132 | 0.042529019 |
| MI | 0.79633351 | 0.49812002 | 0.585511441 | 0.977006056 | 0.513225081 |
| Any Cancer | 0.278468881 | 0.080041396 | 0.144606961 | 0.839610198 | 0.055333432 |
| Lung Cancer | 0.308829703 | 0.501819312 | 0.555589237 | 0.025512668 | 0.789551864 |
| Breast Cancer | 0.62147385 | 0.48011863 | 0.275372927 | 0.711048129 | 0.115249226 |
| Stroke | 0.127884766 | 0.036583822 | 0.044093991 | 0.704660941 | 0.172582214 |
| Mortality | 0.095538396 | 0.147745907 | 0.03168102 | 0.42259479 | 0.016072125 |
| Total | 0.170074575 | 0.598943759 | 0.700230206 | 0.465077856 | 0.380224241 |
| comorbidities | | | | | |
| Disease free | 0.355499334 | 0.248074171 | 0.348466899 | 0.206213659 | 0.076833581 |
| Diabetes | 0.944042235 | 0.875163375 | 0.906947291 | 0.973805231 | 0.096385666 |
| Colorectal | 0.879942769 | 0.929078774 | 0.427381705 | 0.532337389 | 0.442885339 |
| cancer | | | | | |
| Endometrial | 0.673759237 | 0.717487085 | 0.399952655 | 0.866979007 | 0.800261063 |
| cancer | | | | | |
| Leukemia | 0.654138247 | 0.993997507 | 0.885951633 | 0.369771988 | 0.860309417 |
| Arthritis | 0.567343284 | 0.373183051 | 0.898391329 | 0.64309954 | 0.225648364 |
| Thyroid | 0.556679277 | 0.500963122 | 0.273483125 | 0.762508714 | 0.102489185 |
| disease | | | | | |
| Cataract | 0.039784741 | 0.140888851 | 0.179455993 | 0.182873931 | 0.035540473 |

| | Age_prediction | Systems_Clock | DunedinPACE | DNAmGrimAge | PCPhenoAge |
|---|---|---|---|---|---|
| Cognitive | 0.245850616 | 0.460398513 | 0.189287807 | 0.144340115 | 0.470948985 |
| function | | | | | |
| Physical | 0.831219012 | 0.044590365 | 0.134579204 | 0.158117701 | 0.408871895 |
| function | | | | | |
| CHD | 0.881538783 | 0.453276024 | 0.222010047 | 0.181870063 | 0.685687194 |
| MI | 0.995384316 | 0.585876321 | 0.387185653 | 0.149449424 | 0.919856765 |
| Any Cancer | 0.171368594 | 0.433601913 | 0.298972418 | 0.28742935 | 0.058648574 |
| Lung Cancer | 0.741324052 | 0.037587773 | 0.030130079 | 0.014204767 | 0.250581321 |
| Breast Cancer | 0.843923732 | 0.496440754 | 0.567507817 | 0.875148998 | 0.262448881 |
| Stroke | 0.321730252 | 0.82950887 | 0.698647453 | 0.672894008 | 0.220048084 |
| Mortality | 0.012741396 | 0.5381025 | 0.244671082 | 0.240482732 | 0.014838327 |
| Total | 0.088800776 | 0.703811094 | 0.37022797 | 0.873416522 | 0.187622826 |
| comorbidities | | | | | |
| Disease free | 0.707762216 | 0.080618539 | 0.056166744 | 0.022216856 | 0.653549451 |
| Diabetes | 0.42683301 | 0.865039642 | 0.496165755 | 0.531831016 | 0.612060405 |
| Colorectal | 0.874506116 | 0.651560247 | 0.329775639 | 0.545522469 | 0.957378503 |
| cancer | | | | | |
| Endometrial | 0.67492012 | 0.896781361 | 0.916435621 | 0.346371122 | 0.80067832 |
| cancer | | | | | |
| Leukemia | 0.639106137 | 0.994086913 | 0.772290608 | 0.854598265 | 0.965436779 |
| Arthritis | 0.481111217 | 0.746290672 | 0.311343243 | 0.960677322 | 0.530748083 |
| Thyroid | 0.074048211 | 0.291299294 | 0.161915845 | 0.050926647 | 0.156517715 |
| disease | | | | | |
| Cataract | 0.169464669 | 0.07102709 | 0.160476757 | 0.182267331 | 0.177096625 |

| | Horvath1 | Horvath2 | Hannum |
|---|---|---|---|
| Cognitive | 0.791140581 | 0.655796713 | 0.550960447 |
| function | | | |
| Physical | 0.470654664 | 0.619846572 | 0.586771278 |
| function | | | |
| CHD | 0.921449306 | 0.344759066 | 0.288160323 |
| MI | 0.976709131 | 0.314449423 | 0.368583207 |
| Any Cancer | 0.791408314 | 0.494895706 | 0.978319313 |
| Lung Cancer | 0.587806149 | 0.93347941 | 0.321448859 |
| Breast Cancer | 0.079505797 | 0.44091244 | 0.979165206 |
| Stroke | 0.655494252 | 0.011251489 | 0.001836687 |
| Mortality | 0.570951877 | 0.715010826 | 0.769396159 |
| Total | 0.286611511 | 0.183249005 | 0.157642742 |
| comorbidities | | | |
| Disease free | 0.876178441 | 0.625756969 | 0.396139443 |
| Diabetes | 0.455675079 | 0.55192721 | 0.497521372 |
| Colorectal | 0.297976392 | 0.381224413 | 0.454882018 |
| cancer | | | |
| Endometrial | 0.360001671 | 0.199695934 | 0.337137904 |
| cancer | | | |
| Leukemia | 0.237494724 | 0.069988874 | 0.346915299 |
| Arthritis | 0.433512997 | 0.651094069 | 0.506976503 |
| Thyroid | 0.73280625 | 0.288335764 | 0.375675827 |
| disease | | | |
| Cataract | 0.088632352 | 0.164206221 | 0.131276441 |

Our results suggested high levels of specificity of system scores to the expected organ system (FIG. 2 as well as Tables 4 and 5). This was true across baseline and future conditions as well as functional phenotypes and diseases. For functional phenotypes at baseline, the Brain score showed the strongest association with cognitive function (meta z-score=3.51), and the Musculoskeletal score had the strongest association with physical function (meta z-score=8.53). |The Heart score was most strongly associated with time-to-CHD events (meta z-score=8.29) as well as with time to myocardial infarction (meta z-score=6.30). The Lung score was most strongly associated with time to lung cancer (meta z-score=9.69) with the Heart score coming a close second (meta z-score=9.49), which shares risk factors such as smoking with cardiovascular disease. For time to stroke, Heart score (meta z-score=3.40) was the most strongly associated, with the Metabolic score coming a close second (meta z-score=3.35). The blood score was most strongly associated with time to Leukemia (meta z-score=4.88).

TABLE 5
Z-scores for meta-analysis (age and smoking adjusted)

| | Blood | Brain | Inflammation | Heart | Hormone | Immune |
|---|---|---|---|---|---|---|
| Cognitive | 2.698139015 | 3.399211357 | 3.107234097 | 2.357822571 | 1.649931771 | 2.364293791 |
| function | | | | | | |
| Physical | 5.234994385 | 5.779844607 | 7.881921416 | 7.845363439 | 5.185213177 | 3.584781898 |
| function | | | | | | |
| CHD | 3.001946665 | 3.371151264 | 6.326291029 | 6.579236668 | 4.911178629 | 5.134852573 |
| MI | 1.840680363 | 1.931650633 | 4.48170978 | 5.048561717 | 3.652939494 | 4.329626517 |
| Any Cancer | −1.254400476 | −0.757733627 | −0.088811919 | 1.794851633 | 0.286019827 | 1.146163234 |
| Lung Cancer | 1.448908398 | 1.516480354 | 2.508566166 | 4.932574656 | 1.767860766 | 2.511003479 |
| Breast Cancer | −2.520551448 | −1.235116577 | −1.307310504 | −1.412870813 | −3.078891435 | −1.289020306 |
| Stroke | 1.7871408 | 2.659646779 | 2.782523969 | 2.53632064 | 1.542962189 | 2.174502357 |
| Mortality | 6.622210022 | 6.458608882 | 10.66239391 | 11.93212083 | 7.28732607 | 6.933369648 |
| Total | 3.12566849 | 4.491421614 | 7.596105186 | 7.950001994 | 3.952319522 | 3.823513086 |
| comorbidities | | | | | | |
| Disease free | 2.396956288 | 3.027968868 | 2.247321418 | 3.84792547 | 2.238033939 | 1.605767347 |
| Diabetes | 3.585742126 | 4.020953643 | 8.291558824 | 6.947462259 | 5.025985504 | 1.397087499 |
| Colorectal | 1.995868481 | 0.521951771 | 0.533560355 | 0.13364297 | 1.545685361 | 0.438032723 |
| cancer | | | | | | |
| Endometrial | −2.041278649 | −1.622768258 | −1.60108727 | −1.565850592 | −0.972009519 | −0.312748431 |
| cancer | | | | | | |
| Leukemia | 4.687774329 | 3.88853293 | 2.903488601 | 2.736216283 | 2.958329738 | 3.534526662 |
| Arthritis | 1.63958241 | 1.905210714 | 4.494547689 | 3.712512414 | 1.82769185 | 0.851614653 |
| Thyroid | 0.184808594 | −0.055165401 | 1.12011672 | 2.351238441 | 2.761404906 | 0.611259954 |
| disease | | | | | | |
| Cataract | 2.756976477 | 3.038654676 | 2.125753207 | 3.320352161 | 1.954440571 | 1.288919992 |

| | Kidney | Liver | Metabolic | Lung | MusculoSkeletal |
|---|---|---|---|---|---|
| Cognitive | 2.775271157 | 2.130039831 | 2.231221227 | 1.483269676 | 0.890609167 |
| function | | | | | |
| Physical | 5.961226696 | 5.362587465 | 7.187780443 | 4.570833644 | 8.982727589 |
| function | | | | | |
| CHD | 5.227688561 | 3.793606404 | 5.225002268 | 4.295568331 | 5.345135615 |
| MI | 3.715873111 | 2.759319326 | 3.479518456 | 3.51156242 | 3.281673549 |
| Any Cancer | −0.022523013 | −0.582980777 | −0.666529633 | 2.800616832 | 0.366479626 |
| Lung Cancer | 1.860403581 | 1.471612091 | 1.550337887 | 4.630685583 | 0.558560247 |
| Breast Cancer | −1.653820324 | −1.07694179 | −2.039236643 | −1.506542269 | −0.220215968 |
| Stroke | 1.335907624 | 1.983958118 | 3.207696662 | 0.593843186 | 2.384268334 |
| Mortality | 8.80013394 | 6.20377285 | 9.007001171 | 8.993344693 | 7.865907049 |
| Total | 5.625987421 | 4.247530419 | 6.436828929 | 4.419197944 | 7.558846819 |
| comorbidities | | | | | |
| Disease free | 2.232700904 | 2.946374834 | 2.516920403 | 2.297910392 | 2.32179411 |
| Diabetes | 5.09133377 | 3.884660325 | 8.388114796 | 3.855395369 | 10.75986982 |
| Colorectal | 1.246775711 | 1.293562459 | 1.040316972 | 0.054407044 | 1.197441631 |
| cancer | | | | | |
| Endometrial | −0.745492767 | −0.618988424 | −1.872433619 | −1.196720492 | −1.017716736 |
| cancer | | | | | |
| Leukemia | 4.047238534 | 2.330911727 | 3.421174735 | 3.430862898 | 2.410626761 |
| Arthritis | 3.478216217 | 2.17345344 | 2.950332089 | 1.618315852 | 4.971274099 |
| Thyroid | 0.490603978 | −0.049895796 | 0.382633985 | 1.928387328 | 1.052441345 |
| disease | | | | | |
| Cataract | 2.27902371 | 3.475760691 | 2.82343829 | 1.570326954 | 1.488774937 |

| | Age_prediction | Systems_Clock | DunedinPACE | DNAmGrimAge | PCPhenoAge |
|---|---|---|---|---|---|
| Cognitive | 1.533950321 | 2.404824018 | 1.633187218 | 1.56450899 | 2.903353688 |
| function | | | | | |
| Physical | 5.637066358 | 8.141015355 | 8.71435293 | 5.856465971 | 7.895656788 |
| function | | | | | |
| CHD | 3.095897961 | 6.648043812 | 5.767639845 | 5.807126678 | 5.769131025 |
| MI | 1.193526742 | 5.000318059 | 4.642569379 | 4.345627477 | 3.423574442 |
| Any Cancer | 0.858349844 | 1.770487324 | 1.130689826 | 4.067408616 | 1.131224985 |
| Lung Cancer | 2.047055014 | 4.821284198 | 2.6349896 | 5.946010109 | 2.750108066 |
| Breast Cancer | −1.795030003 | −1.522732339 | 0.586294917 | −0.984021911 | −1.382580398 |
| Stroke | 0.743775388 | 2.497128391 | 2.921060099 | 1.232694698 | 2.283719973 |
| Mortality | 7.189524186 | 12.07984787 | 8.890852659 | 12.22217204 | 10.71585594 |
| Total | 4.00401551 | 7.989525311 | 9.26612934 | 7.425011358 | 7.364961136 |
| comorbidities | | | | | |
| Disease free | 2.878668997 | 3.821747976 | 4.078034126 | 3.816149351 | 3.095967417 |
| Diabetes | 2.597263042 | 7.253682642 | 9.556879473 | 7.705750562 | 7.280045495 |
| Colorectal | 1.865993209 | 0.33448036 | 0.507432338 | 1.621539732 | 1.226898537 |
| cancer | | | | | |
| Endometrial | −0.600256362 | −1.591114101 | −0.570125992 | 0.170986862 | −1.009232804 |
| cancer | | | | | |
| Leukemia | 3.735667891 | 3.149728265 | 0.940724073 | 0.572281683 | 2.583169142 |
| Arthritis | 1.5088354 | 3.772465501 | 5.079863721 | 2.357445461 | 3.010865759 |
| Thyroid | 1.630076808 | 2.267984084 | 1.626106556 | 2.940695841 | 1.740090841 |
| disease | | | | | |
| Cataract | 2.899429047 | 3.286220057 | 2.430654045 | 2.394200554 | 2.609411423 |

| | Horvath1 | Horvath2 | Hannum |
|---|---|---|---|
| Cognitive | 0.719904585 | 0.71857594 | 2.083840352 |
| function | | | |
| Physical | 2.892425439 | 4.170878498 | 3.922158074 |
| function | | | |
| CHD | 1.188028972 | 0.829920072 | 2.581363071 |
| MI | 0.44131703 | −0.588461323 | 1.380068659 |
| Any Cancer | 1.233222015 | 0.247086271 | −0.106323932 |
| Lung Cancer | 2.469544712 | 1.139536428 | 0.967206581 |
| Breast Cancer | −1.065296173 | −3.007643665 | −3.794733774 |
| Stroke | 0.37446172 | 0.55943517 | 2.670414353 |
| Mortality | 2.998329915 | 4.928470321 | 5.875165666 |
| Total | 2.580352567 | 3.022684937 | 3.514097888 |
| comorbidities | | | |
| Disease free | 3.60195235 | 1.300753295 | 1.95123673 |
| Diabetes | 2.103522384 | 1.493178592 | 2.214912516 |
| Colorectal | 0.26117645 | 1.246731666 | 1.646495114 |
| cancer | | | |
| Endometrial | 1.417367825 | −0.619672138 | 0.508067535 |
| cancer | | | |
| Leukemia | 1.842128649 | 2.980293204 | 2.897337222 |
| Arthritis | 0.610910453 | 0.52126033 | 0.096513965 |
| Thyroid | 1.819349991 | 1.307313952 | 1.872400853 |
| disease | | | |
| Cataract | 0.292890389 | 1.539209276 | 1.592830583 |

In almost all conditions and disease outcomes, we observed the expected directionality—with increased age indicators associated with increased risk. The one exception to this was reproductive organ cancers, in which we observed negative associations with all of the systems. Hormone (meta z-value=−3.04) and Blood (meta z-value=−2.22) were the most negatively associated with breast and endometrial cancer, respectively.

For diseases at baseline, Musculoskeletal was most strongly associated with diabetes (meta z-value=10.90) and arthritis (meta z-value=5.15). Hormone had the strongest association with thyroid disease (meta z-value=2.65) and for cataract, both Heart (meta z-value=3.16) and Liver (meta z-value=3.12) had the strongest associations. Last of all, the total comorbidities variable at baseline was most strongly associated with Inflammation (meta z-value=7.65).

### Compared to Existing Clocks, System Scores Better Capture the Multifactorial Nature of Aging

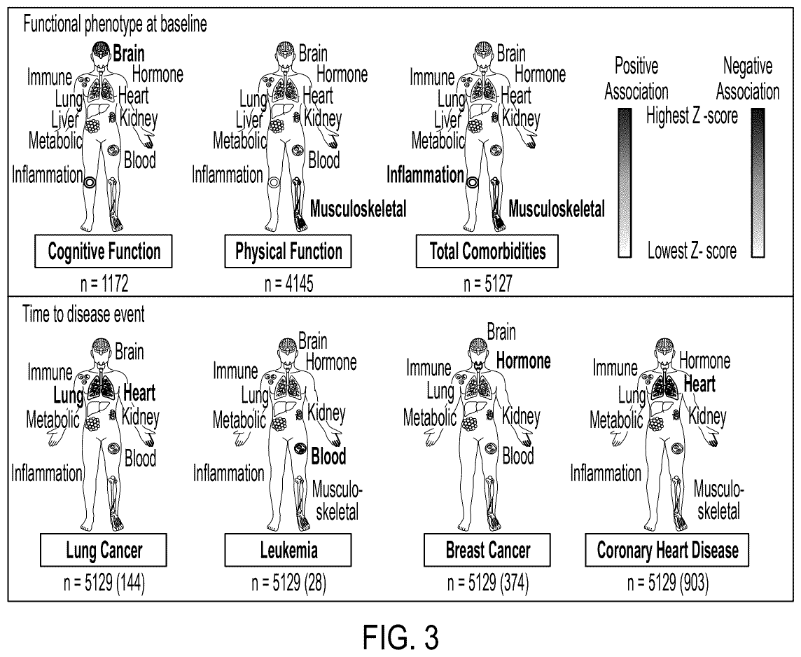

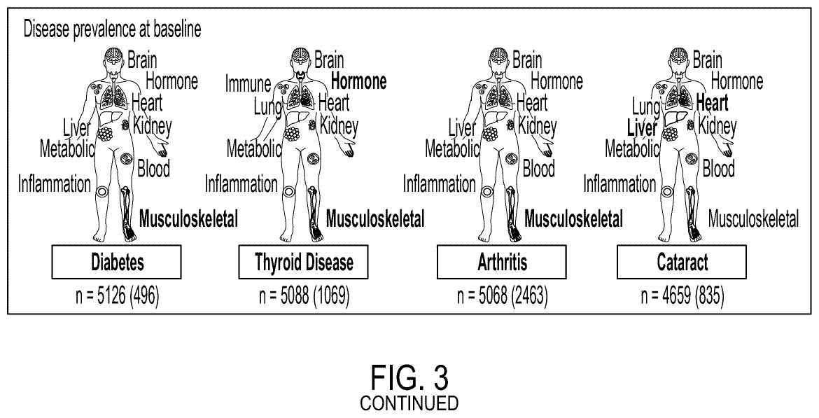

We compared system scores and Systems Age to previously trained epigenetic clocks using meta z-scores (FIG. 3, Tables 3-6), estimated in the same manner as was done for system scores in the previous section (FIG. 2). We focused on three prominent epigenetic clocks that have previously been demonstrated to be strongly associated with aging outcomes: PCPhenoAge, DNAmGrimAge and DunedinPACE.

TABLE 6
P-values for heterogeneity in meta-analysis (age and smoking adjusted)

| | Blood | Brain | Inflammation | Heart | Hormone | Immune |
|---|---|---|---|---|---|---|
| Cognitive | 0.149845596 | 0.354627794 | 0.31787378 | 0.713951753 | 0.604157603 | 0.058734756 |
| function | | | | | | |
| Physical | 0.036090531 | 0.270965239 | 0.658016225 | 0.133718111 | 0.439577734 | 0.283272188 |
| function | | | | | | |
| CHD | 0.976680968 | 0.742093272 | 0.309773356 | 0.64967247 | 0.681927365 | 0.653479961 |
| MI | 0.787469224 | 0.479816156 | 0.418651883 | 0.853713966 | 0.835506356 | 0.366003265 |
| Any Cancer | 0.530160088 | 0.240199586 | 0.21920052 | 0.30142766 | 0.321213326 | 0.364044656 |
| Lung Cancer | 0.770551992 | 0.358944403 | 0.146276072 | 0.298132908 | 0.494540288 | 0.879464481 |
| Breast Cancer | 0.501962883 | 0.668080765 | 0.351075216 | 0.340100647 | 0.204169493 | 0.454351388 |
| Stroke | 0.296002191 | 0.305457314 | 0.787409984 | 0.654896132 | 0.231512974 | 0.123880799 |
| Mortality | 0.915116367 | 0.32641965 | 0.066513852 | 0.72011904 | 0.261715282 | 0.426805728 |
| Total | 0.389061198 | 0.515272133 | 0.169135377 | 0.47644213 | 0.353756673 | 0.306776415 |
| comorbidities | | | | | | |
| Disease free | 0.781874881 | 0.512311692 | 0.459178078 | 0.246299417 | 0.243051496 | 0.32074141 |
| Diabetes | 0.857196773 | 0.667271929 | 0.838374728 | 0.609746715 | 0.193164946 | 0.809830465 |
| Colorectal | 0.982829914 | 0.594233733 | 0.789731966 | 0.61504083 | 0.713871763 | 0.309492859 |
| cancer | | | | | | |
| Endometrial | 0.945863578 | 0.401278038 | 0.413070198 | 0.865136245 | 0.557017361 | 0.52360101 |
| cancer | | | | | | |
| Leukemia | 0.93410385 | 0.942296904 | 0.187120829 | 0.924466697 | 0.061905454 | 0.468747792 |
| Arthritis | 0.807755001 | 0.752008218 | 0.725208277 | 0.398062157 | 0.530487289 | 0.560282507 |
| Thyroid | 0.3022335 | 0.255981858 | 0.334227716 | 0.590099083 | 0.342562598 | 0.896361258 |
| disease | | | | | | |
| Cataract | 0.420529231 | 0.373980647 | 0.099027306 | 0.105813444 | 0.179500105 | 0.303707344 |

| | Kidney | Liver | Metabolic | Lung | MusculoSkeletal |
|---|---|---|---|---|---|
| Cognitive | 0.185330546 | 0.075861292 | 0.247987578 | 0.170221049 | 0.963679448 |
| function | | | | | |
| Physical | 0.403317743 | 0.502370762 | 0.092644995 | 0.886612934 | 0.209119506 |
| function | | | | | |
| CHD | 0.965778505 | 0.781733885 | 0.588932995 | 0.859895591 | 0.104630857 |
| MI | 0.879107472 | 0.48627808 | 0.647886487 | 0.990609171 | 0.562051849 |
| Any Cancer | 0.384254191 | 0.107901033 | 0.151782124 | 0.700873893 | 0.060853182 |
| Lung Cancer | 0.44132088 | 0.492940785 | 0.744216762 | 0.266784356 | 0.849867325 |
| Breast Cancer | 0.625544153 | 0.445919118 | 0.262922641 | 0.59224672 | 0.116298546 |
| Stroke | 0.152182302 | 0.043523102 | 0.048359242 | 0.566589051 | 0.225866736 |
| Mortality | 0.273939435 | 0.213856547 | 0.053671874 | 0.739593211 | 0.06397787 |
| Total | 0.11185487 | 0.547293539 | 0.642629528 | 0.415104861 | 0.357773993 |
| comorbidities | | | | | |
| Disease free | 0.443307249 | 0.263724008 | 0.423757642 | 0.332754427 | 0.131806085 |
| Diabetes | 0.961120567 | 0.941180748 | 0.944943782 | 0.767990248 | 0.108970199 |
| Colorectal | 0.913768032 | 0.934892179 | 0.467468867 | 0.733259457 | 0.444897083 |
| cancer | | | | | |
| Endometrial | 0.700688732 | 0.745012059 | 0.327285848 | 0.946257058 | 0.834819005 |
| cancer | | | | | |
| Leukemia | 0.684368468 | 0.998633784 | 0.936934471 | 0.671113104 | 0.875629803 |
| Arthritis | 0.617089164 | 0.276573474 | 0.935461402 | 0.138691116 | 0.13185603 |
| Thyroid | 0.548367381 | 0.426451926 | 0.3517976 | 0.76468082 | 0.119214849 |
| disease | | | | | |
| Cataract | 0.082795114 | 0.430707082 | 0.273122999 | 0.427516536 | 0.025750179 |

| | Age_prediction | Systems_Clock | DunedinPACE | DNAmGrimAge | PCPhenoAge |
|---|---|---|---|---|---|
| Cognitive | 0.278619993 | 0.607971831 | 0.121930199 | 0.150221138 | 0.425530923 |
| function | | | | | |
| Physical | 0.704363687 | 0.200929511 | 0.129918483 | 0.342624683 | 0.412532608 |
| function | | | | | |
| CHD | 0.952290353 | 0.656936134 | 0.173790902 | 0.228224746 | 0.775768388 |
| MI | 0.991890762 | 0.878510472 | 0.438900444 | 0.206178366 | 0.932282678 |
| Any Cancer | 0.220984936 | 0.244537339 | 0.135250756 | 0.118114538 | 0.074764261 |
| Lung Cancer | 0.642716933 | 0.302381502 | 0.095171741 | 0.157546503 | 0.317558991 |
| Breast Cancer | 0.783805407 | 0.346320751 | 0.43582289 | 0.628186305 | 0.248324554 |
| Stroke | 0.433113379 | 0.691215355 | 0.593179767 | 0.767900951 | 0.220242672 |
| Mortality | 0.063795064 | 0.58143872 | 0.216190272 | 0.509173006 | 0.054363149 |
| Total | 0.083257416 | 0.473613281 | 0.189435143 | 0.190101055 | 0.147604337 |
| comorbidities | | | | | |
| Disease free | 0.752558692 | 0.246468646 | 0.161871736 | 0.267819667 | 0.682013359 |
| Diabetes | 0.488144449 | 0.596465589 | 0.610636341 | 0.14248703 | 0.698107261 |
| Colorectal | 0.918790425 | 0.738927468 | 0.3174161 | 0.558922508 | 0.972825647 |
| cancer | | | | | |
| Endometrial | 0.732706876 | 0.86757155 | 0.94248452 | 0.71991728 | 0.807150401 |
| cancer | | | | | |
| Leukemia | 0.44573026 | 0.963975213 | 0.813120078 | 0.89703592 | 0.929195611 |
| Arthritis | 0.587288582 | 0.324544871 | 0.063099829 | 0.210963877 | 0.553157668 |
| Thyroid | 0.067048441 | 0.596876327 | 0.333040722 | 0.187860127 | 0.166267613 |
| disease | | | | | |
| Cataract | 0.201368939 | 0.086595767 | 0.19768695 | 0.304977276 | 0.277625041 |

| | Horvath1 | Horvath2 | Hannum |
|---|---|---|---|
| Cognitive | 0.884592919 | 0.517868675 | 0.285501297 |
| function | | | |
| Physical | 0.490886783 | 0.64340972 | 0.672911256 |
| function | | | |
| CHD | 0.937335765 | 0.170724858 | 0.259221675 |
| MI | 0.943121528 | 0.162555297 | 0.28394155 |
| Any Cancer | 0.843599575 | 0.279410082 | 0.91073431 |
| Lung Cancer | 0.596373648 | 0.960415227 | 0.255889401 |
| Breast Cancer | 0.090778264 | 0.380870321 | 0.969217998 |
| Stroke | 0.612757443 | 0.015611534 | 0.002422332 |
| Mortality | 0.701180416 | 0.600879514 | 0.810508991 |
| Total | 0.245163959 | 0.107348882 | 0.083945532 |
| comorbidities | | | |
| Disease free | 0.959339858 | 0.642176066 | 0.436452133 |
| Diabetes | 0.426360353 | 0.506285016 | 0.44719919 |
| Colorectal | 0.380219341 | 0.336568192 | 0.471492685 |
| cancer | | | |
| Endometrial | 0.154877894 | 0.219544848 | 0.35003478 |
| cancer | | | |
| Leukemia | 0.2488365 | 0.065665081 | 0.323402343 |
| Arthritis | 0.443089853 | 0.705679545 | 0.448631924 |
| Thyroid | 0.7877542 | 0.275329338 | 0.272162178 |
| disease | | | |
| Cataract | 0.183771117 | 0.214536272 | 0.133005912 |

The most relevant system scores outperformed or were comparable to existing epigenetic clocks for 6 of the 10 diseases and conditions, including diabetes at baseline (Musculoskeletal meta z-score 10.90; DunedinPACE 8.96), time to leukemia (Blood meta z-score 4.88; PCPhenoage 2.74), cognitive function (Brain meta z-score 3.51; PCPhenoAge 2.95), cataract at baseline (Liver meta z-score 3.12; PCPhenoAge 2.59), time to breast cancer (Hormone absolute meta z-score 3.01; PCPhenoage 1.32), and time to endometrial cancer (Blood absolute meta z-score 2.22; PCPhenoage 1.19). In 3 of the diseases and conditions the system scores were marginally better-arthritis at baseline (Musculoskeletal meta z-score 5.15; DunedinPACE 4.96), time to myocardial infarction (Heart meta z-score 6.30; DNAmGrimAge 6.09), and time to CHD (Heart meta z-score 8.29; DNAmGrimAge 8.11). For the remaining 4 diseases and conditions, the clocks were a close second to existing clocks-physical function (musculoskeletal meta z-score 9.46; DunedinPACE 9.47), time to stroke (Heart meta z-score 3.40; DunedinPACE3.45), thyroid disease at baseline (Hormone meta z-score 2.65; DNAmGrimAge 2.92), and time to lung cancer (Lung meta z-score 9.69; DNAmGrimAge 12.11). For the three whole body metrics, system specific scores were second-best including for time to death or Mortality (Heart meta z-score 15.17; DNAmGrimAge 16.81), total comorbidities at baseline (Inflammation meta z-score 7.65; DunedinPACE 9.08), and disease free at baseline (Heart meta z-score 3.98; DunedinPACE 4.28).

### Systems Age: The Golden Mean

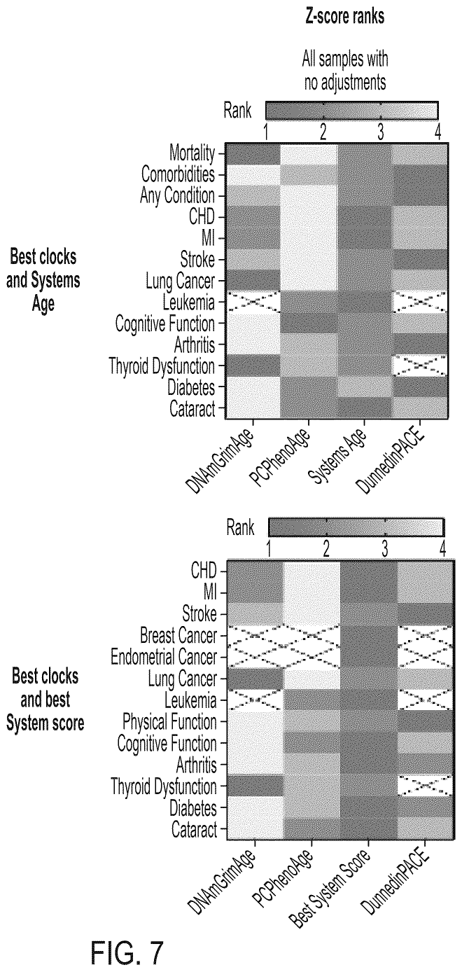

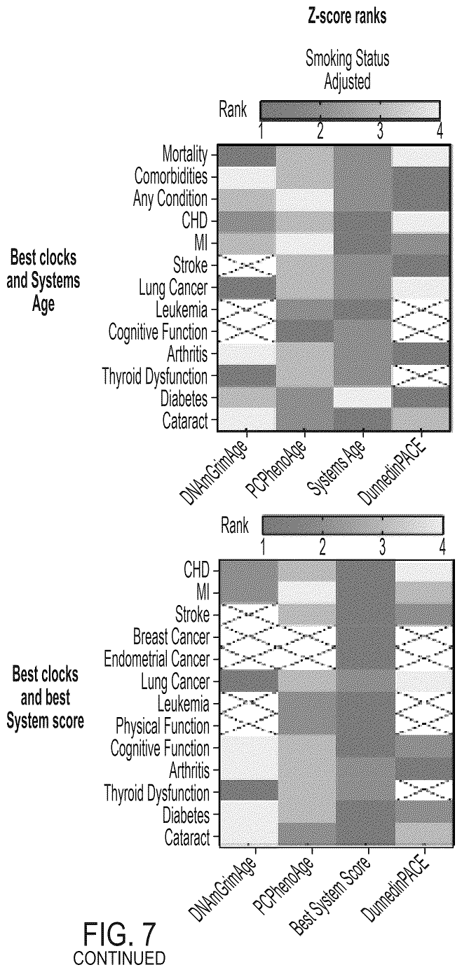

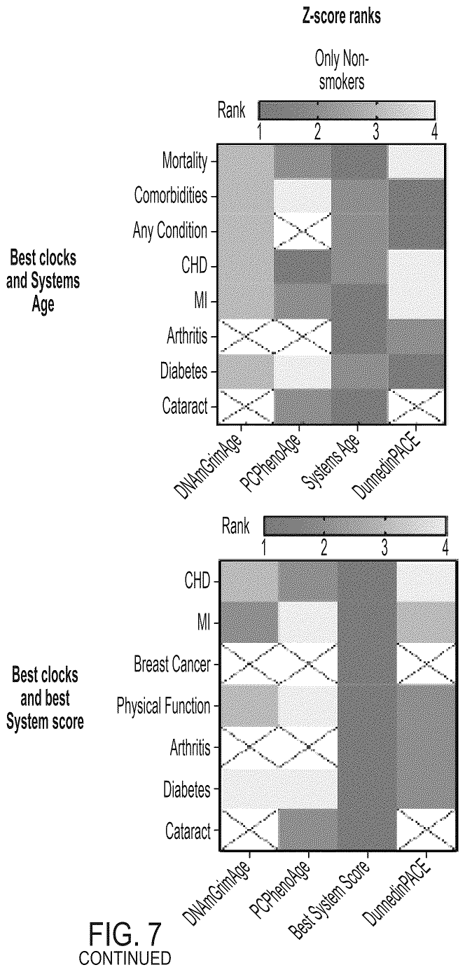

When training clocks, each may be biased towards predicting specific aspects of aging based on the combination of variables and datasets used for training. Since each systems score showed superior or equivalent associations with specific diseases and aging phenotypes, we hypothesized that combining them into a single Systems Age score would lead to a more uniform prediction across all diseases and aging phenotypes. Indeed, we found that Systems Age was not biased to a specific dimension of aging and performed relatively well across a variety of diseases and conditions. Of the 14 different conditions we tested, every clock showed significant associations (FIGS. 2 and 7), Systems Age had the strongest associations of all clocks for four conditions, including cataract (Systems Age 3.17; PCPhenoAge 2.59), CHD (Systems Age 8.27; DNAmGrimAge 8.11), myocardial infarction (Systems Age 6.17; DNAmGrimAge 6.09), and leukemia (Systems Age 2.84; PCPhenoAge 2.74). For 9 of the conditions, Systems Age was second best, as in the case for time to stroke (Systems Age 3.32; DunedinPACE 3.45), disease free at baseline (Systems Age 3.98; DunnedinPACE 4.28), physical function (Systems Age 9.09; DunedinPACE 9.47), cognitive function (Systems Age 2.64; PCPhenoAge 2.95), time to death (Systems Age 15.1; DNAmGrimAge 16.81), total comorbidities at baseline (Systems Age 7.55; DunnedinPACE 9.08), thyroid disease at baseline (Systems Age 2.34; DNAmGrimAge 2.92), arthritis at baseline (Systems Age 3.38; DNAmGrimAge 4.96), and time to lung cancer (Systems Age 9.12; DNAmGrimAge 12.19). DunedinPACE and DNAmGrimAge did perform best with a few of the conditions (3 and 5 resp.) but last or second to last in most others (10 and 8 resp.), indicating they were biased towards certain dimensions of aging more than others. Overall, this suggests the Systems Age training paradigm enables more uniform and unbiased prediction across many dimensions of aging compared to existing state-of-the-art clocks.

### Capturing Aging Signal Beyond Smoking

Smoking is well known to affect DNA methylation and epigenetic clocks, 21 as well as disease incidence (especially cardiopulmonary diseases and cancer), aging phenotypes, and mortality. To determine how much of the system scores signal was related to smoking, we calculated meta z-scores for these clocks while adjusting for smoking status. For example, the Metabolic system score was strongly associated with stroke, and this association changed minimally when adjusting for smoking status (meta z-score 3.46 as compared to meta z-score 3.32 when adjusted for smoking status). On the other hand, GrimAge's association with time to stroke decreased and was no longer significant when adjusting for smoking (meta z-score 2.79 as compared to meta z-score 1.23 when adjusted for smoking status). Reduced associations with CHD and time to myocardial infarction were observed across all clocks after adjusting for smoking (as expected given that smoking is a major risk factor for heart disease), but Systems Age and Heart score retain much of their association after smoking status adjustment for (Heart 8.29 vs 6.57, Systems Age 8.84 vs 6.64, DNAmGrimAge 8.11 vs 5.80; meta Z-score in myocardial infarction before and after smoking status adjusted for Heart 6.32 vs 5.04, Systems Age 6.17 vs 4.99, DNAmGrimAge 6.09 vs 4.34). Similar impacts of smoking were seen for time to lung cancer and time to death. For other diseases and aging phenotypes, Systems Age and system scores retained their prediction after adjusting for smoking, indicating they captured epigenetic signals beyond just smoking. We also performed an analysis among never smokers (FIG. 7 as well as Tables 7 and 8) finding that system scores performed the best across all the conditions in which at least one score was significant.

TABLE 7
Z-scores for meta-analysis (only non-smokers)

| | Blood | Brain | Inflammation | Heart | Hormone | Immune |
|---|---|---|---|---|---|---|
| Cognitive | 1.11871608 | 1.518733622 | 0.636815188 | 0.762584047 | −0.318194317 | 0.77967801 |
| function | | | | | | |
| Physical | 2.046633499 | 2.416316191 | 4.073640052 | 4.850860097 | 2.776855947 | 1.246626399 |
| function | | | | | | |
| CHD | 3.229350837 | 3.538090077 | 5.520253145 | 4.384414079 | 4.238367545 | 4.352453112 |
| MI | 2.406658972 | 2.366891962 | 3.977103232 | 3.228806129 | 3.642342049 | 3.833828361 |
| Any Cancer | −1.378532044 | −1.366391671 | −1.710537823 | −1.870612048 | 0.089388412 | −0.014683353 |
| Lung Cancer | −0.322263905 | −0.211689183 | −0.255834896 | 0.210402164 | −1.190989746 | 0.318963208 |
| Breast Cancer | −1.168694364 | −0.402554805 | −0.54041947 | −0.864205538 | −1.684499684 | −0.992594275 |
| Stroke | 0.815157853 | 1.474572195 | 1.384225512 | 1.394432859 | 0.36019566 | 1.467869862 |
| Mortality | 3.036945067 | 3.550426207 | 7.08080407 | 6.84558522 | 3.819118671 | 4.838605232 |
| Total | 1.34444284 | 2.30933111 | 4.393151011 | 5.600476682 | 2.139242045 | 2.618669974 |
| comorbidities | | | | | | |
| Disease free | 0.711162869 | 1.925284329 | 1.902775742 | 2.807133463 | 1.86952525 | 1.571356531 |
| Diabetes | 2.724648787 | 3.280072672 | 7.056453958 | 6.944597767 | 3.959548955 | 2.787872322 |
| Colorectal | 2.41345286 | 0.880687984 | 0.761783353 | −0.257555404 | 2.040754529 | 0.459235612 |
| cancer | | | | | | |
| Endometrial | 0.192581537 | −0.422374816 | −0.457316025 | 0.423832242 | 0.169316281 | 1.314462914 |
| cancer | | | | | | |
| Leukemia | 1.051821387 | 0.969503892 | 0.386067443 | 1.24632988 | 0.359274935 | 2.146618422 |
| Arthritis | 0.049182747 | 0.43849251 | 2.07138202 | 2.355591583 | 0.813392805 | 0.072399533 |
| Thyroid | 0.088918737 | −0.558638519 | 0.132917039 | 0.809377498 | 1.517858931 | −0.447666505 |
| disease | | | | | | |
| Cataract | 2.26851677 | 2.66542745 | 2.117963815 | 2.680990111 | 2.057620352 | 1.134125411 |

| | Kidney | Liver | Metabolic | Lung | MusculoSkeletal |
|---|---|---|---|---|---|
| Cognitive | 0.956909464 | 0.539591481 | 0.319210849 | 0.129603955 | −0.51958225 |
| function | | | | | |
| Physical | 2.621870497 | 1.863564593 | 3.139411427 | 2.554850778 | 5.698595652 |
| function | | | | | |
| CHD | 4.642361157 | 4.178373642 | 4.716780318 | 2.631018657 | 3.951118099 |
| MI | 3.707712483 | 3.61963913 | 3.373822697 | 2.317962301 | 2.052437516 |
| Any Cancer | −1.437707537 | −1.267542826 | −1.537461808 | −1.040207915 | −0.381495303 |
| Lung Cancer | −0.992970666 | −1.029402002 | −0.551298394 | 0.245918471 | −0.667467661 |
| Breast Cancer | −1.331339227 | −0.633138203 | −1.53744017 | −1.548186052 | 0.435350337 |
| Stroke | 0.601632851 | 1.435013669 | 1.598860035 | 0.65979872 | 0.990760328 |
| Mortality | 5.018727396 | 3.850818152 | 5.20289285 | 5.493376636 | 4.422920796 |
| Total | 3.126074113 | 2.558170296 | 3.314436172 | 3.231341728 | 4.287867117 |
| comorbidities | | | | | |
| Disease free | 1.525443802 | 1.715303929 | 1.316184389 | 2.109828074 | 0.867869686 |
| Diabetes | 4.927739322 | 3.845064801 | 6.950060088 | 3.575759721 | 7.890798762 |
| Colorectal | 1.538769071 | 1.498533463 | 1.500883836 | −0.2208159 | 1.063940554 |
| cancer | | | | | |
| Endometrial | 0.445108106 | 0.738864948 | 0.141539531 | −0.122237907 | −0.601844883 |
| cancer | | | | | |
| Leukemia | 1.291340686 | 0.910498626 | 1.610962392 | 1.550676133 | 1.382885422 |
| Arthritis | 1.399005061 | 0.95577416 | 0.631738138 | 1.168693272 | 2.975525304 |
| Thyroid | −0.212776447 | −0.52471769 | −0.682077122 | 0.015462397 | 0.257766585 |
| disease | | | | | |
| Cataract | 2.039349822 | 2.809467144 | 2.277893395 | 1.866671328 | 1.222048799 |

| | Age_prediction | Systems_Clock | DunedinPACE | DNAmGrimAge | PCPhenoAge |
|---|---|---|---|---|---|
| Cognitive | −0.476787689 | 0.64609295 | 0.641784265 | 0.863996953 | 0.568954562 |
| function | | | | | |
| Physical | 3.103658743 | 4.921948748 | 4.604308674 | 4.376530498 | 4.27807025 |
| function | | | | | |
| CHD | 3.540550424 | 4.587278094 | 3.549037986 | 4.108287107 | 4.906764143 |
| MI | 2.514825003 | 3.372688449 | 2.674380101 | 3.279441 | 3.367734431 |
| Any Cancer | 0.445469045 | −1.745501759 | −1.271811644 | −1.422683411 | −0.086366358 |
| Lung Cancer | 1.27739296 | 0.083165664 | −1.767845816 | −0.538720095 | 0.284426554 |
| Breast Cancer | −1.510390102 | −0.98893665 | −0.387836931 | −1.230939086 | −1.195226134 |
| Stroke | 0.556822338 | 1.373580028 | 1.465121817 | 0.070871714 | 1.366912811 |
| Mortality | 3.876296189 | 6.953529183 | 3.200745798 | 5.69798433 | 6.431632421 |
| Total | 2.023922496 | 5.487953547 | 6.055802049 | 4.812287923 | 4.353247138 |
| comorbidities | | | | | |
| Disease free | 0.7483957 | 2.704879046 | 2.249193892 | 2.919965315 | 1.639801288 |
| Diabetes | 2.659453415 | 7.011243058 | 7.605277329 | 7.392668088 | 6.075598575 |
| Colorectal | 2.459717529 | 0.047359466 | 0.684536318 | 1.33824992 | 1.654893678 |
| cancer | | | | | |
| Endometrial | 1.812442392 | 0.372283263 | 1.079482126 | 1.538345187 | 0.583557728 |
| cancer | | | | | |
| Leukemia | 1.066561387 | 1.377873095 | 1.125498988 | −0.154176367 | 1.258715572 |
| Arthritis | 0.596659396 | 2.328292886 | 1.86326468 | 0.12096569 | 1.507085941 |
| Thyroid | 1.660156736 | 0.712510643 | 1.295263918 | 1.125197288 | 0.850068584 |
| disease | | | | | |
| Cataract | 2.146074978 | 2.71527648 | 1.206733616 | 1.634250975 | 2.077999033 |

| | Horvath1 | Horvath2 | Hannum |
|---|---|---|---|
| Cognitive | −0.492398085 | 0.63481368 | 0.820505575 |
| function | | | |
| Physical | 1.515772721 | 3.145967458 | 1.537237236 |
| function | | | |
| CHD | 0.987016676 | 0.896773139 | 2.044550048 |
| MI | 0.911813294 | 0.144644958 | 1.615163338 |
| Any Cancer | 0.6498665 | 0.700878644 | −1.028391523 |
| Lung Cancer | 0.751838281 | 0.571715485 | 0.283545916 |
| Breast Cancer | −1.166062716 | −2.216681643 | −2.493130622 |
| Stroke | 0.157261643 | 0.415389146 | 2.867227019 |
| Mortality | 1.89830927 | 2.187198417 | 2.99293219 |
| Total | 1.665875888 | 1.589079914 | 1.348337083 |
| comorbidities | | | |
| Disease free | 2.068834484 | 0.702948181 | 1.136684745 |
| Diabetes | 2.092959976 | 3.150764685 | 2.87547055 |
| Colorectal | 1.079819806 | 2.147528309 | 1.435814368 |
| cancer | | | |
| Endometrial | 1.985325907 | 0.549078029 | 0.877205494 |
| cancer | | | |
| Leukemia | 1.496889556 | 1.318086654 | 2.122154451 |
| Arthritis | 0.535890317 | 1.039255698 | −0.335589079 |
| Thyroid | 2.026421189 | 1.3246971 | 1.791764866 |
| disease | | | |
| Cataract | 0.018716791 | 1.490089622 | 1.030213763 |

TABLE 8
P-values for heterogeneity in meta-analysis (non-smokers only)

| | Blood | Brain | Inflammation | Heart | Hormone | Immune |
|---|---|---|---|---|---|---|
| Cognitive | 0.41347311 | 0.377947062 | 0.904555523 | 0.871366803 | 0.820015959 | 0.611692103 |
| function | | | | | | |
| Physical | 0.116336652 | 0.431042952 | 0.399237305 | 0.375006235 | 0.314719366 | 0.319349472 |
| function | | | | | | |
| CHD | 0.727073615 | 0.795639822 | 0.214771142 | 0.745088045 | 0.218189734 | 0.367200844 |
| MI | 0.675993479 | 0.921586869 | 0.28950505 | 0.886561906 | 0.301497365 | 0.544736508 |
| Any Cancer | 0.561471855 | 0.536225748 | 0.177983577 | 0.454753932 | 0.748301455 | 0.541370811 |
| Lung Cancer | 0.057860962 | 0.034123181 | 0.207345346 | 0.423663621 | 0.528249908 | 0.597435032 |
| Breast Cancer | 0.488989069 | 0.573486771 | 0.238963241 | 0.202672264 | 0.509502183 | 0.709065545 |
| Stroke | 0.183067158 | 0.50151279 | 0.874779819 | 0.845085604 | 0.764843312 | 0.817628719 |
| Mortality | 0.282249539 | 0.292219532 | 0.022184788 | 0.429989853 | 0.717664581 | 0.688870769 |
| Total | 0.230233038 | 0.251476289 | 0.078213743 | 0.221210754 | 0.002844189 | 0.128451142 |
| comorbidities | | | | | | |
| Disease free | 0.196375924 | 0.648757907 | 0.028133232 | 0.181517905 | 0.076083866 | 0.639130954 |
| Diabetes | 0.912217291 | 0.648072818 | 0.643503612 | 0.5768459 | 0.264386341 | 0.798452515 |
| Colorectal | 0.789030397 | 0.4805965 | 0.659647698 | 0.589236537 | 0.71060144 | 0.858265308 |
| cancer | | | | | | |
| Endometrial | 0.888162403 | 0.215899045 | 0.616931178 | 0.752260939 | 0.924984934 | 0.51570644 |
| cancer | | | | | | |
| Leukemia | 0.303465188 | 0.628668773 | 0.735088259 | 0.681239529 | 0.092288412 | 0.542392037 |
| Arthritis | 0.497381578 | 0.612917959 | 0.419174496 | 0.286345481 | 0.408442653 | 0.680186119 |
| Thyroid | 0.313817138 | 0.510881179 | 0.936209645 | 0.733039189 | 0.146068225 | 0.739593931 |
| disease | | | | | | |
| Cataract | 0.124212321 | 0.026166669 | 0.028418501 | 0.019751292 | 0.163471173 | 0.134765901 |

| | Kidney | Liver | Metabolic | Lung | MusculoSkeletal |
|---|---|---|---|---|---|
| Cognitive | 0.865612253 | 0.297288008 | 0.681634183 | 0.907022287 | 0.571927699 |
| function | | | | | |
| Physical | 0.377574878 | 0.489356853 | 0.198143343 | 0.83419328 | 0.6593847 |
| function | | | | | |
| CHD | 0.506592263 | 0.803750203 | 0.4882428 | 0.968980595 | 0.380804892 |
| MI | 0.658969476 | 0.784465238 | 0.68240641 | 0.917814373 | 0.332515844 |
| Any Cancer | 0.676144273 | 0.271589486 | 0.557826354 | 0.806740333 | 0.068224088 |
| Lung Cancer | 0.122631123 | 0.112333426 | 0.336568542 | 0.924079808 | 0.223618322 |
| Breast Cancer | 0.771182318 | 0.773540932 | 0.525118579 | 0.076422528 | 0.01883923 |
| Stroke | 0.329266536 | 0.335454585 | 0.132774006 | 0.609012725 | 0.299714994 |
| Mortality | 0.124657889 | 0.156071554 | 0.046850205 | 0.99977393 | 0.259385818 |
| Total | 0.029362631 | 0.435489119 | 0.219929181 | 0.698426901 | 0.127438333 |
| comorbidities | | | | | |
| Disease free | 0.205008189 | 0.325659541 | 0.201740566 | 0.204218962 | 0.07699437 |
| Diabetes | 0.774323059 | 0.899059918 | 0.867991345 | 0.910964294 | 0.53762483 |
| Colorectal | 0.800192271 | 0.708042729 | 0.318138626 | 0.911263179 | 0.334765586 |
| cancer | | | | | |
| Endometrial | 0.54243568 | 0.913424867 | 0.509633775 | 0.764327774 | 0.871721085 |
| cancer | | | | | |
| Leukemia | 0.768052579 | 0.725703624 | 0.90537969 | 0.555628684 | 0.905628184 |
| Arthritis | 0.579936826 | 0.824785131 | 0.750731671 | 0.772959557 | 0.052567575 |
| Thyroid | 0.704334642 | 0.870506031 | 0.891395019 | 0.465515813 | 0.317312605 |
| disease | | | | | |
| Cataract | 0.083713512 | 0.135839374 | 0.03099481 | 0.024894101 | 0.095750467 |

| | Age_prediction | Systems_Clock | DunedinPACE | DNAmGrimAge | PCPhenoAge |
|---|---|---|---|---|---|
| Cognitive | 0.74241996 | 0.898257276 | 0.301951339 | 0.619840397 | 0.818086487 |
| function | | | | | |
| Physical | 0.544674356 | 0.416522698 | 0.148306916 | 0.207841505 | 0.429447366 |
| function | | | | | |
| CHD | 0.469752183 | 0.722123535 | 0.778210483 | 0.905069876 | 0.401345462 |
| MI | 0.44497587 | 0.864996941 | 0.839474252 | 0.933730529 | 0.568375197 |
| Any Cancer | 0.641757907 | 0.439255689 | 0.263653139 | 0.53092686 | 0.472682851 |
| Lung Cancer | 0.035269786 | 0.393134437 | 0.660321879 | 0.837641403 | 0.085408309 |
| Breast Cancer | 0.792083051 | 0.167524779 | 0.25440093 | 0.42802795 | 0.347520375 |
| Stroke | 0.483183426 | 0.85001776 | 0.497295355 | 0.711943764 | 0.412212492 |
| Mortality | 0.236836273 | 0.366022981 | 0.468786192 | 0.411344343 | 0.103621509 |
| Total | 0.379398761 | 0.215527514 | 0.004475394 | 0.197810756 | 0.03617064 |
| comorbidities | | | | | |
| Disease free | 0.581719796 | 0.133289505 | 0.327328419 | 0.648497385 | 0.398292258 |
| Diabetes | 0.507593948 | 0.606230924 | 0.852945253 | 0.575781166 | 0.721734468 |
| Colorectal | 0.462431136 | 0.664620926 | 0.249977472 | 0.396752371 | 0.606175315 |
| cancer | | | | | |
| Endometrial | 0.77958395 | 0.739557264 | 0.713497101 | 0.788786395 | 0.836084991 |
| cancer | | | | | |
| Leukemia | 0.508412126 | 0.613467735 | 0.829328024 | 0.343843617 | 0.543383361 |
| Arthritis | 0.603333319 | 0.279108196 | 0.058897834 | 0.16345479 | 0.416092857 |
| Thyroid | 0.167092639 | 0.731379762 | 0.261955489 | 0.215265996 | 0.34558989 |
| disease | | | | | |
| Cataract | 0.467040073 | 0.01388495 | 0.032004334 | 0.451236213 | 0.14260602 |

| | Horvath1 | Horvath2 | Hannum |
|---|---|---|---|
| Cognitive | 0.800906964 | 0.415389564 | 0.637027501 |
| function | | | |
| Physical | 0.804517681 | 0.956680867 | 0.407739795 |
| function | | | |
| CHD | 0.747213871 | 0.453830884 | 0.246821413 |
| MI | 0.40254718 | 0.340849949 | 0.15593404 |
| Any Cancer | 0.916418909 | 0.948507133 | 0.776134543 |
| Lung Cancer | 0.117653118 | 0.274640403 | 0.022116317 |
| Breast Cancer | 0.560772439 | 0.699693733 | 0.448549356 |
| Stroke | 0.788989786 | 0.069954401 | 0.134479876 |
| Mortality | 0.814045307 | 0.555847574 | 0.292722216 |
| Total | 0.815700636 | 0.424599627 | 0.300758595 |
| comorbidities | | | |
| Disease free | 0.44288292 | 0.271293485 | 0.113761589 |
| Diabetes | 0.559104691 | 0.714773183 | 0.988454509 |
| Colorectal | 0.296695629 | 0.317985203 | 0.566696698 |
| cancer | | | |
| Endometrial | 0.252274857 | 0.32352807 | 0.252650125 |
| cancer | | | |
| Leukemia | 0.39025636 | 0.356574793 | 0.548098721 |
| Arthritis | 0.309201861 | 0.38618963 | 0.347048441 |
| Thyroid | 0.744467111 | 0.316644621 | 0.409915447 |
| disease | | | |
| Cataract | 0.672055952 | 0.558614583 | 0.092227218 |

### System Scores Capture Distinct Dimensions of Aging

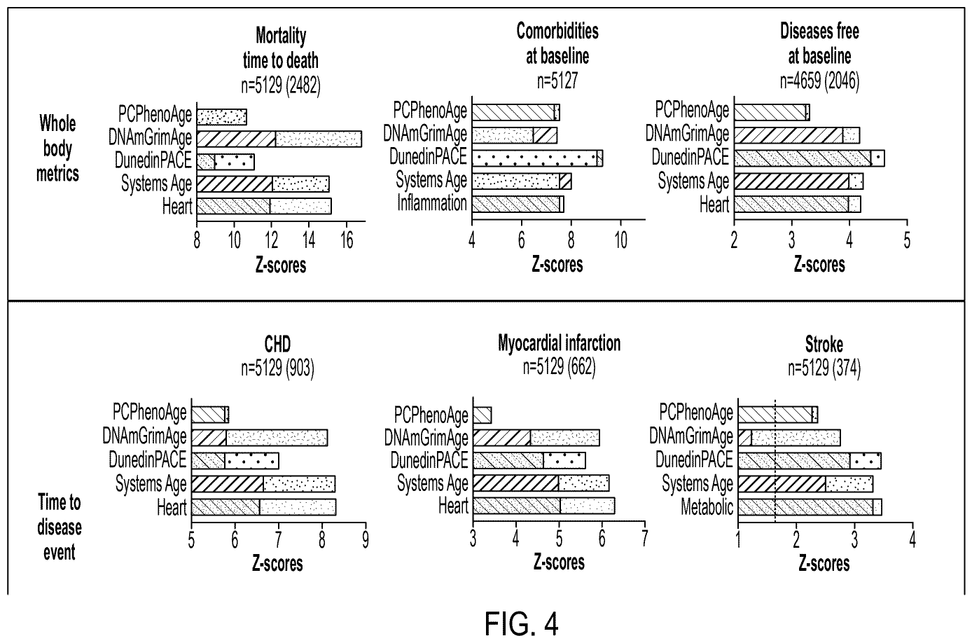

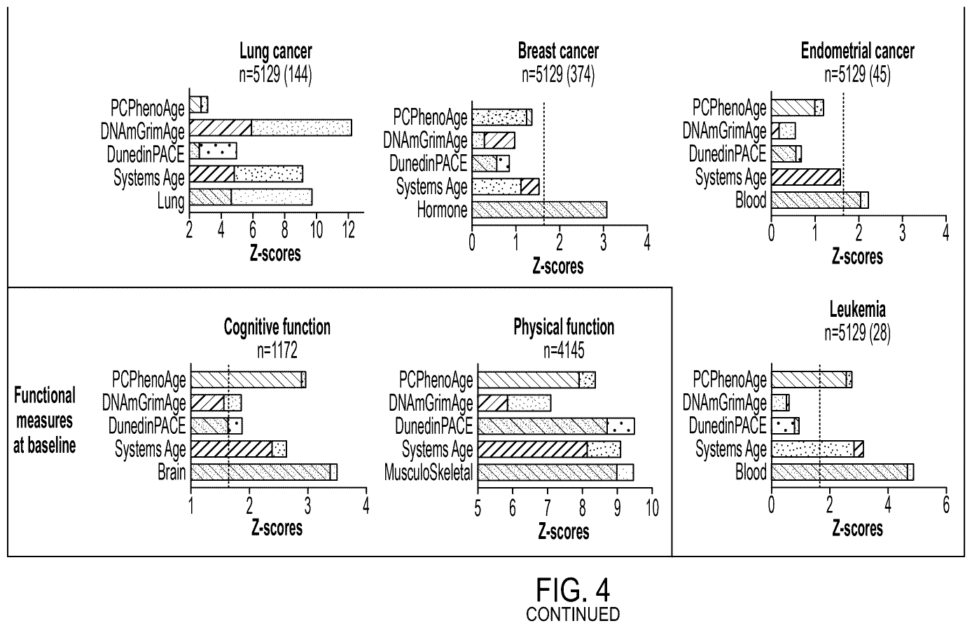

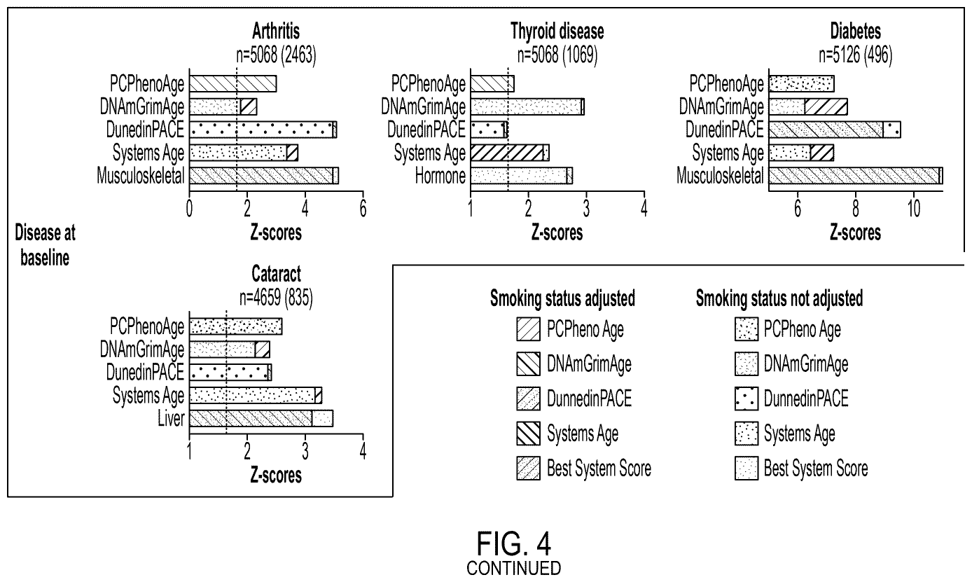

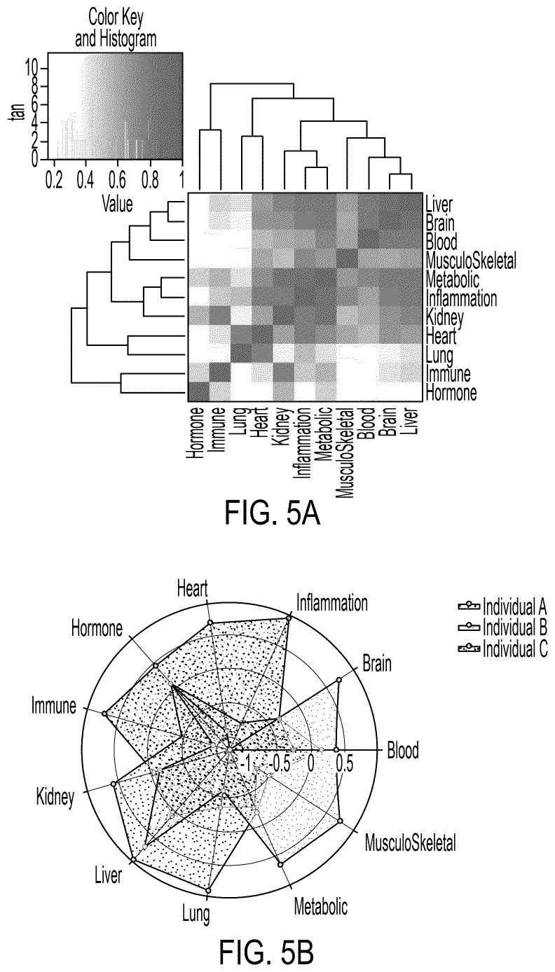

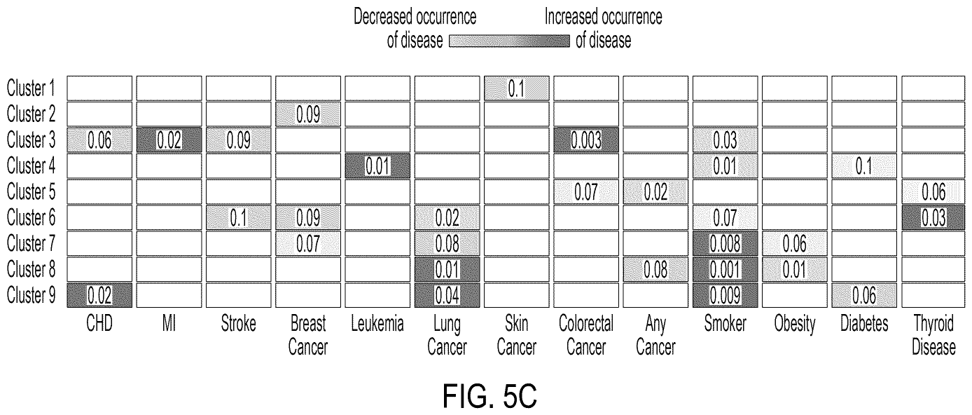

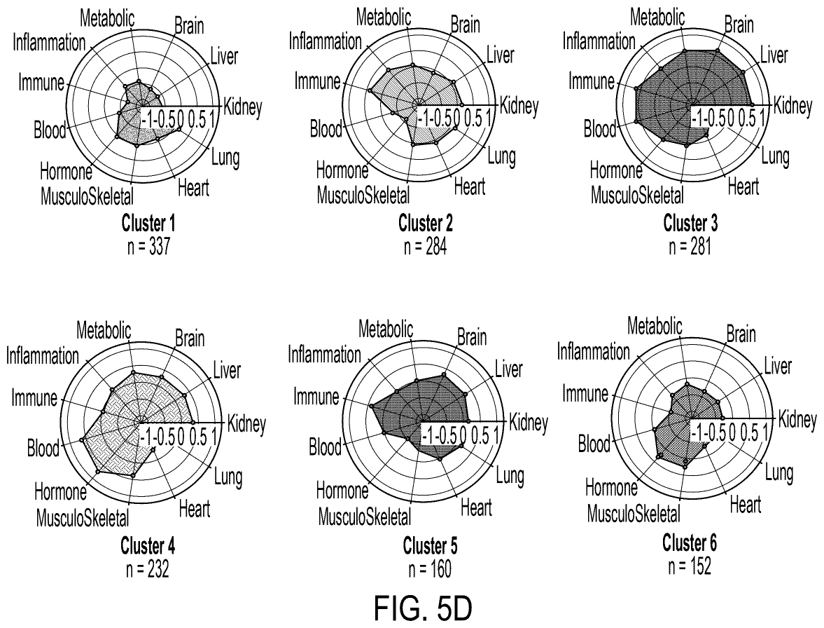

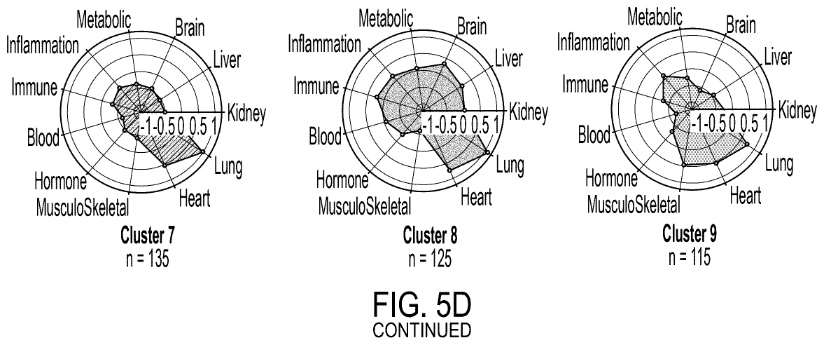

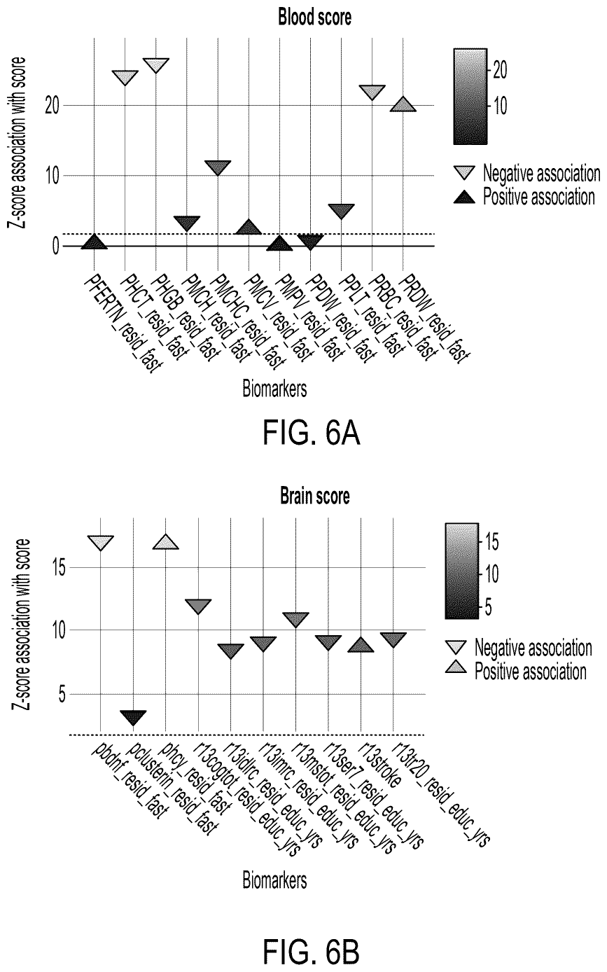

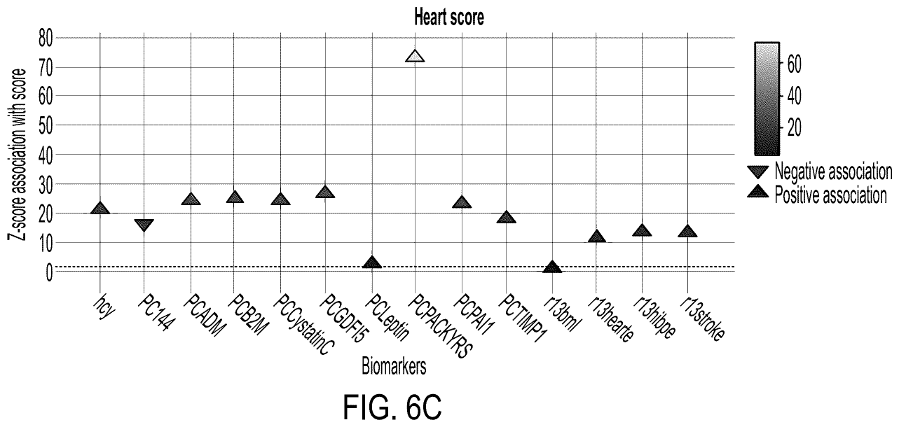

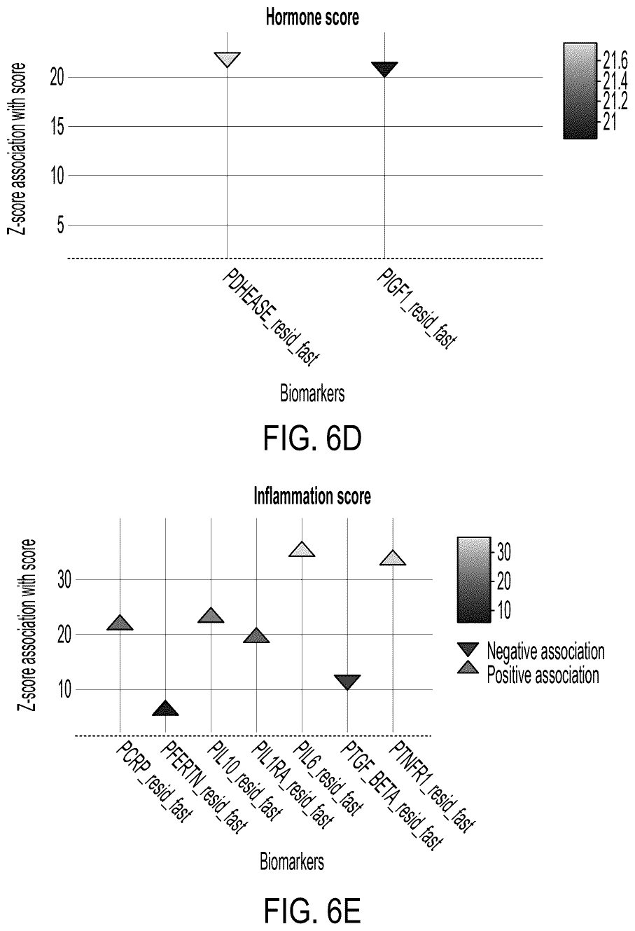

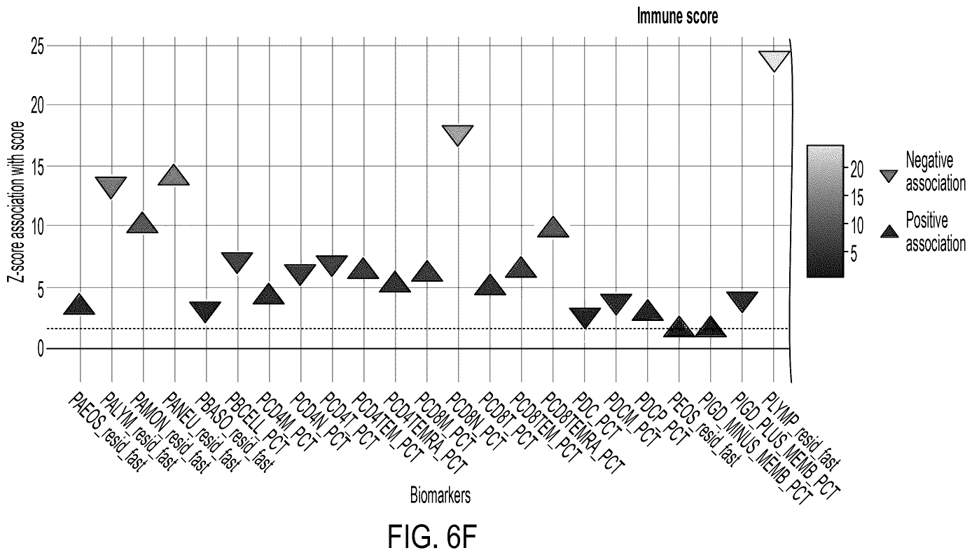

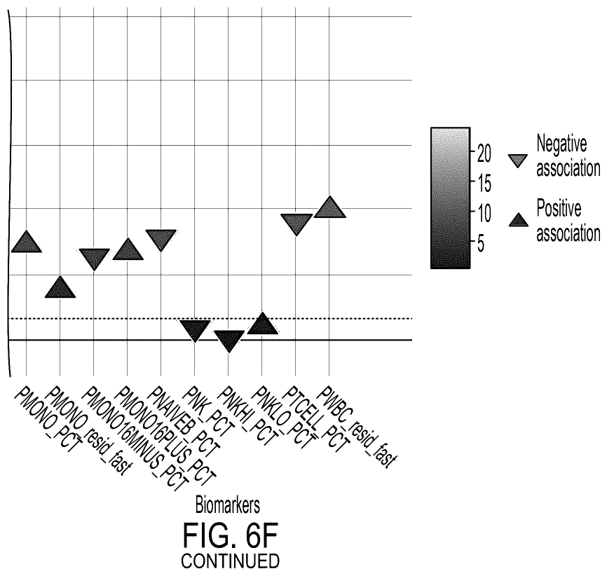

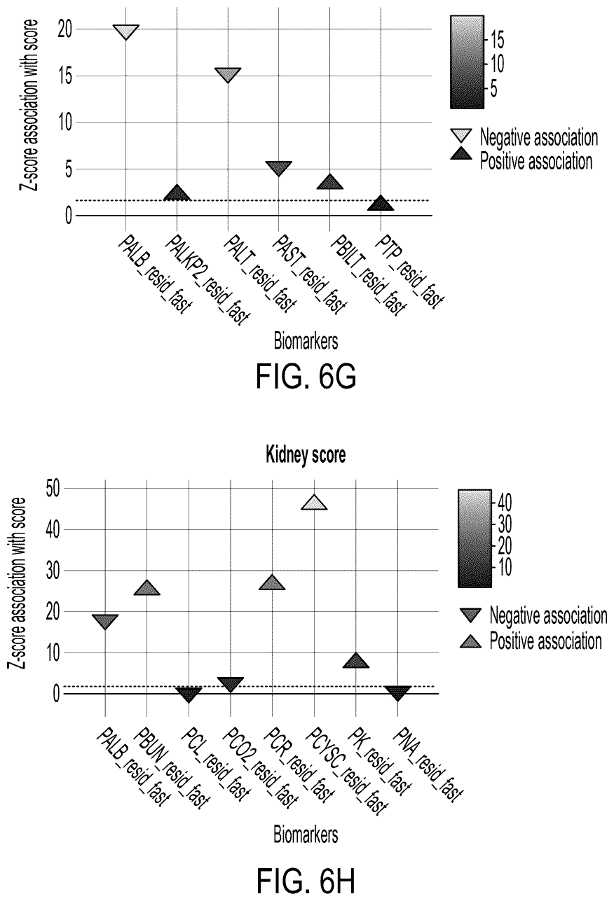

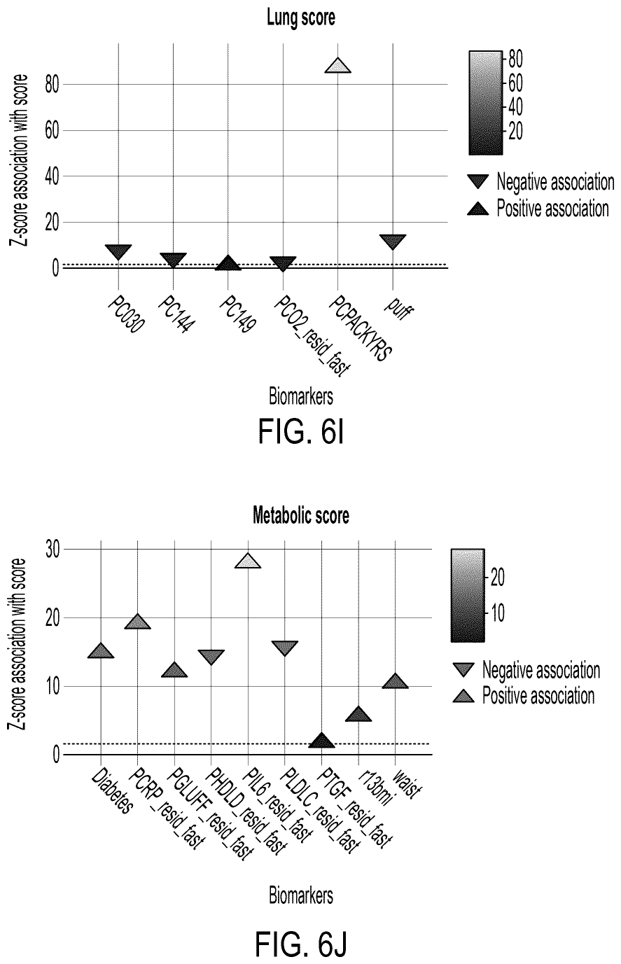

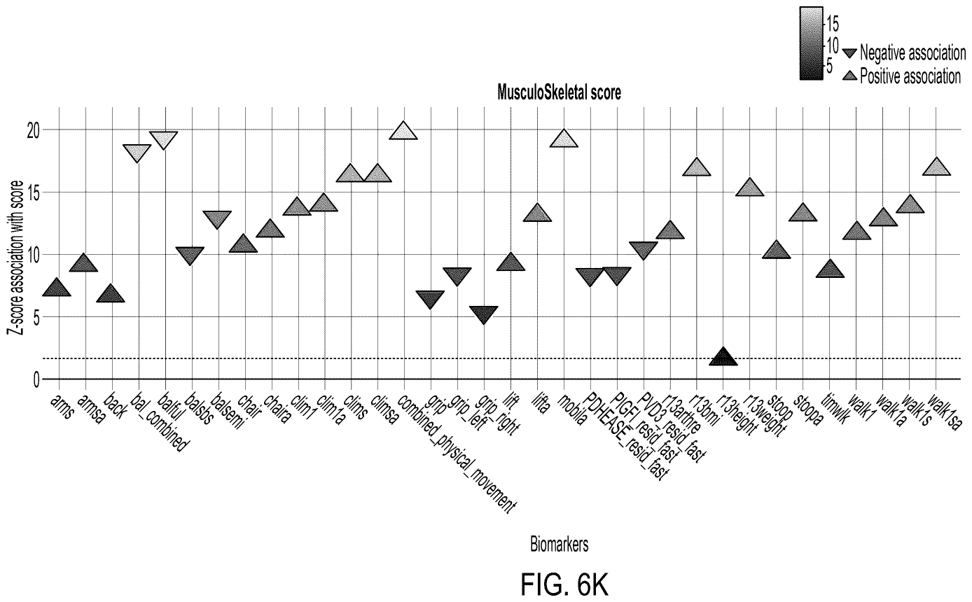

Systems do not work independently of each other. Some systems are closely related, sharing many associations with a given disease, condition, or aging phenotype. Indeed, looking at correlations between different age-adjusted system scores across the WHI cohorts (FIG. 4A and Table 9), we found some systems to be highly correlated with each other, such as Heart and Lung (r=0.759), or Inflammation and Musculoskeletal (r=0.716). Hierarchical clustering revealed that Heart and Lung formed a cluster, Liver, Brain, and Blood formed a second cluster, and Metabolic, Inflammation, and Kidney formed a third cluster. The latter two clusters formed a super-cluster that also included Heart and Musculoskeletal. Given these patterns, we hypothesized that there is predictive value in examining not just heterogeneity within a system or across systems, but also to test whether individuals can be grouped based on their systems scores to generate aging subtypes with distinct predisposition to aging related diseases and conditions.

TABLE 9
Correlation between system scores

| | Blood | Brain | Inflammation | Heart | Hormone | Immune |
|---|---|---|---|---|---|---|
| Blood | 1 | 0.807588953 | 0.632236757 | 0.553582926 | 0.331667315 | 0.285133103 |
| Brain | 0.807588953 | 1 | 0.773711361 | 0.675148811 | 0.253758738 | 0.42556976 |
| Cytokine | 0.632236757 | 0.773711361 | 1 | 0.788676909 | 0.363881138 | 0.504766663 |
| Heart | 0.553582926 | 0.675148811 | 0.788676909 | 1 | 0.256246227 | 0.436378273 |
| Hormone | 0.331667315 | 0.253758738 | 0.363881138 | 0.256246227 | 1 | 0.480020405 |
| Immune | 0.285133103 | 0.42556976 | 0.504766663 | 0.436378273 | 0.480020405 | 1 |
| Kidney | 0.591417759 | 0.685438174 | 0.79121833 | 0.62573013 | 0.569713009 | 0.776987565 |
| Liver | 0.797379154 | 0.864663155 | 0.801394187 | 0.643938896 | 0.289862987 | 0.47325181 |
| Metabolic | 0.738528325 | 0.804681272 | 0.849869592 | 0.703655913 | 0.488822721 | 0.572611834 |
| Lung | 0.321205318 | 0.384102757 | 0.544412238 | 0.759474382 | 0.16672346 | 0.322384468 |
| Musculo- | 0.624157976 | 0.604582445 | 0.716789032 | 0.613368527 | 0.232951645 | 0.165736602 |
| Skeletal | | | | | | |

| | | | | | Musculo- |
|---|---|---|---|---|---|
| | Kidney | Liver | Metabolic | Lung | Skeletal |
| Blood | 0.591417759 | 0.797379154 | 0.738528325 | 0.321205318 | 0.624157976 |
| Brain | 0.685438174 | 0.864663155 | 0.804681272 | 0.384102757 | 0.604582445 |
| Cytokine | 0.79121833 | 0.801394187 | 0.849869592 | 0.544412238 | 0.716789032 |
| Heart | 0.62573013 | 0.643938896 | 0.703655913 | 0.759474382 | 0.613368527 |
| Hormone | 0.569713009 | 0.289862987 | 0.488822721 | 0.16672346 | 0.232951645 |
| Immune | 0.776987565 | 0.47325181 | 0.572611834 | 0.322384468 | 0.165736602 |
| Kidney | 1 | 0.74534667 | 0.845902751 | 0.391154309 | 0.535458085 |
| Liver | 0.74534667 | 1 | 0.815340968 | 0.437989213 | 0.64204629 |
| Metabolic | 0.845902751 | 0.815340968 | 1 | 0.433643318 | 0.663603241 |
| Lung | 0.391154309 | 0.437989213 | 0.433643318 | 1 | 0.350335801 |
| Musculo- | 0.535458085 | 0.64204629 | 0.663603241 | 0.350335801 | 1 |
| Skeletal | | | | | |

To test this, we first examined whether there existed individuals with the same chronological age and overall Systems Age yet different age-accelerated system scores. One example of this was observed for three individuals from the HRS dataset (FIG. 4B), with the same chronological age, Systems Age, gender, and race. However, they had entirely different patterns of systems scores.

To test whether these patterns of system scores constituted biologically relevant aging subtypes, we clustered individuals using adaptive hierarchical clustering in the WHI EMPC cohort. This analysis identified 9 unique groups or clusters, with each cluster showing distinct patterns across the different system aging scores, as well as different associations with the prevalence or future occurrence of certain diseases (FIGS. 4C-D, Tables 10 and 11). For example, Cluster 8 and 9 were found to have a high mean Lung aging score (Lung age accelerations 1.06 and 0.71) and a higher prevalence of future lung cancer events (pval: 0.01, 0.04). These groups also had an overrepresentation of smokers (pval: 0.001, 0.009), indicating they captured smoking pathophysiology. As expected, their distinct clustering stemmed from other pathophysiology: Group 8 had fewer obese individuals (p=0.01) while Group 9 was enriched for future CHD events (p=0.02). All of this indicated that the 2 groups were capturing distinct aging subtypes within smokers. At the same time, Cluster 3 demonstrated an increased prevalence of MI (p=0.02) yet it showed low Lung aging and decreased prevalence of smokers (p=0.03). It also demonstrated increased Metabolic (average age acceleration 0.55) and Inflammation (average age acceleration 0.34) aging. Thus, while Cluster 3 and 9 are both at risk for cardiovascular diseases, the risk stems from different sources (smoking vs. metabolic and inflammatory aging). Overall, the system's scores were capturing relevant aging subtypes that had distinct behavioral and genetic patterns predisposing individuals to certain types of aging phenotypes and diseases.

TABLE 10
Fisher's P-value (Greater)

| | COG_VAR | PHYS_VAR | CHD | MI | ANYCANCER | LUNG |
|---|---|---|---|---|---|---|
| 1 | 0.787670588 | 0.858725512 | 0.873893637 | 0.782107209 | 0.534946711 | 0.217503543 |
| 2 | 0.505160024 | 0.369136696 | 0.851551407 | 0.878439414 | 0.459619939 | 0.751825229 |
| 3 | 0.494259114 | 0.394871789 | 0.068626666 | 0.021820586 | 0.203453137 | 0.929150289 |
| 4 | 0.202233645 | 0.300441974 | 0.8268731 | 0.860208624 | 0.333914277 | 0.936864277 |
| 5 | 0.62601661 | 0.444715206 | 0.847080646 | 0.848325375 | 0.985916031 | 0.91895307 |
| 6 | 0.771412912 | 0.64572168 | 0.217094141 | 0.144825707 | 0.889168744 | 1 |
| 7 | 0.178414644 | 0.682353507 | 0.356260375 | 0.589707387 | 0.749172745 | 0.085500318 |
| 8 | 0.962816031 | 0.646871619 | 0.273902927 | 0.373653425 | 0.088336608 | 0.010955024 |
| 9 | 0.386909458 | 0.689968729 | 0.023515433 | 0.114523378 | 0.283930715 | 0.04565691 |

| | BREAST | STROKE | OBESITY | DIAB | COLORECTAL | ENDMTRL |
|---|---|---|---|---|---|---|
| 1 | 0.81497621 | 0.658129887 | 0.339712718 | 0.505958425 | 0.812923726 | 0.968620796 |
| 2 | 0.093448638 | 0.653183115 | 0.117565317 | 0.416703715 | 0.790079516 | 0.385376092 |
| 3 | 0.258579847 | 0.092842239 | 0.251357499 | 0.698862292 | 0.003359883 | 0.378452915 |
| 4 | 0.406592059 | 0.822296288 | 0.13054916 | 0.941587746 | 0.7648086 | 0.468552539 |
| 5 | 0.838021939 | 0.297989151 | 0.738089628 | 0.506399342 | 0.986623287 | 0.522586111 |
| 6 | 0.095065804 | 0.109160501 | 0.6564923 | 0.652411421 | 0.789731208 | 1 |
| 7 | 0.968588771 | 0.711777531 | 0.9515629 | 0.897332418 | 0.339205116 | 1 |
| 8 | 0.36286125 | 0.259597321 | 0.990687693 | 0.762188309 | 0.857940875 | 0.402096093 |
| 9 | 0.508690538 | 0.82257569 | 0.218549601 | 0.060322963 | 0.236412357 | 0.144479072 |

| | LEUKEMIA | MELANOMA | ARTHRIT | THYROID | SMOKEVR |
|---|---|---|---|---|---|
| 1 | 0.495220182 | 0.104851429 | 0.53435343 | 0.751000966 | 0.234556181 |
| 2 | 0.742367764 | 0.518285204 | 0.467158367 | 0.641569874 | 0.118002339 |
| 3 | 1 | 0.873520756 | 0.569227103 | 0.848874987 | 0.976989368 |
| 4 | 0.011521726 | 0.589125552 | 0.42963295 | 0.503972203 | 0.9992832 |
| 5 | 0.544597691 | 0.873240019 | 0.414768608 | 0.960656606 | 0.885433928 |
| 6 | 1 | 0.860016177 | 0.696280581 | 0.031252209 | 0.941703028 |
| 7 | 1 | 0.276520348 | 0.68736387 | 0.137555666 | 0.008322306 |
| 8 | 1 | 0.803833784 | 0.50725075 | 0.328434158 | 0.001944219 |
| 9 | 1 | 0.453750724 | 0.132740791 | 0.209964518 | 0.009452427 |

TABLE 10B
Fisher's P-value (Lesser)

| | COG_VAR | PHYS_VAR | CHD | MI | ANYCANCER | LUNG |
|---|---|---|---|---|---|---|
| 1 | 0.341622853 | 0.182264978 | 0.181871284 | 0.306702671 | 0.521955546 | 0.867146694 |
| 2 | 0.644458732 | 0.692569094 | 0.215205653 | 0.195134636 | 0.600249065 | 0.39727928 |
| 3 | 0.654779823 | 0.668935541 | 0.955539274 | 0.988024583 | 0.835984207 | 0.159953684 |
| 4 | 0.887425355 | 0.759859272 | 0.253433028 | 0.228433776 | 0.723562026 | 0.159499962 |
| 5 | 0.576711914 | 0.640341525 | 0.24606855 | 0.2659497 | 0.024676148 | 0.223408492 |
| 6 | 0.42996327 | 0.444243799 | 0.858236617 | 0.917315377 | 0.158054791 | 0.022037627 |
| 7 | 0.916986544 | 0.410873087 | 0.753470467 | 0.567144522 | 0.327236034 | 0.964947462 |
| 8 | 0.164745404 | 0.452057029 | 0.821692076 | 0.760020252 | 0.94018668 | 0.996566712 |
| 9 | 0.796052645 | 0.410497734 | 0.992132398 | 0.941225388 | 0.786448909 | 0.983829095 |

| | BREAST | STROKE | OBESITY | DIAB | COLORECTAL | ENDMTRL |
|---|---|---|---|---|---|---|
| 1 | 0.253809918 | 0.43738189 | 0.698027675 | 0.579412514 | 0.308248238 | 0.148844872 |
| 2 | 0.937872066 | 0.450378404 | 0.902915582 | 0.669546032 | 0.348003524 | 0.802397282 |
| 3 | 0.809307429 | 0.940838988 | 0.783319271 | 0.38988303 | 0.998680298 | 0.807596045 |
| 4 | 0.688084947 | 0.269717145 | 0.893462633 | 0.100503338 | 0.393828917 | 0.759685063 |
| 5 | 0.258293173 | 0.802262297 | 0.313998184 | 0.611607863 | 0.074000939 | 0.754737775 |
| 6 | 0.944077402 | 0.937943189 | 0.40215872 | 0.470522886 | 0.404483137 | 0.212049946 |
| 7 | 0.074117575 | 0.432202271 | 0.068652108 | 0.185515137 | 0.816909345 | 0.250930757 |
| 8 | 0.752076914 | 0.840299467 | 0.015210065 | 0.362570115 | 0.339638818 | 0.845900333 |
| 9 | 0.630966784 | 0.314270901 | 0.827648616 | 0.968088215 | 0.888401968 | 0.959421937 |

| | LEUKEMIA | MELANOMA | ARTHRIT | THYROID | SMOKEVR |
|---|---|---|---|---|---|
| 1 | 0.787356687 | 0.953653744 | 0.507770157 | 0.298758781 | 0.795348175 |
| 2 | 0.630348922 | 0.689318842 | 0.577602878 | 0.419428488 | 0.901918034 |
| 3 | 0.296303245 | 0.316626884 | 0.476211 | 0.193136974 | 0.030279352 |
| 4 | 0.998035207 | 0.648062717 | 0.618265086 | 0.564174045 | 0.001137195 |
| 5 | 0.827583892 | 0.399875973 | 0.641092272 | 0.062947582 | 0.146499773 |
| 6 | 0.508512773 | 0.426605061 | 0.359549275 | 0.979837655 | 0.078997409 |
| 7 | 0.547243136 | 0.893114402 | 0.372386261 | 0.902682199 | 0.994407601 |
| 8 | 0.571534234 | 0.526942105 | 0.558523405 | 0.747086582 | 0.998784164 |
| 9 | 0.597016662 | 0.808443237 | 0.899232183 | 0.848705688 | 0.993792636 |

TABLE 11
Mean age-accelerated score for each cluster

| | Kidney | Liver | Brain | Metabolic | Inflammation | Immune |
|---|---|---|---|---|---|---|
| 1 | −0.59098063 | −0.643693283 | −0.604552494 | −0.411067944 | −0.356554799 | −0.708341815 |
| 2 | 0.196393443 | 0.1155274 | −0.098752443 | 0.055173172 | 0.218895721 | 0.378556259 |
| 3 | 0.667068784 | 0.695513875 | 0.69398006 | 0.557006526 | 0.343903858 | 0.687088744 |
| 4 | 0.313083929 | 0.299773908 | 0.266224719 | 0.301384631 | 0.105235358 | −0.009004468 |
| 5 | 0.151146615 | 0.2576529 | 0.338562327 | 0.047186724 | 0.022832151 | 0.418222873 |
| 6 | −0.330313039 | −0.303545596 | −0.326636681 | −0.176246825 | −0.281638315 | −0.529865905 |
| 7 | −0.489395921 | −0.558800092 | −0.452300553 | −0.370961143 | −0.196659674 | −0.283378987 |
| 8 | 0.048167064 | 0.141805189 | 0.337505811 | 0.08715411 | 0.177974337 | 0.261871351 |
| 9 | −0.271415097 | −0.460101122 | −0.58149169 | −0.244572196 | 0.125257355 | −0.300739278 |

| | | | Musculo- | | |
|---|---|---|---|---|---|
| | Blood | Hormone | Skeletal | Heart | Lung |
| 1 | −0.4198822 | 0.064019786 | 0.046748664 | −0.096827426 | 0.136216897 |
| 2 | −0.373911497 | −0.566061906 | 0.130474654 | 0.13602284 | 0.170618149 |
| 3 | 0.66884632 | 0.253730143 | 0.086651204 | −0.156751736 | −0.574350911 |
| 4 | 0.647859249 | 0.731254007 | 0.380024632 | −0.300115096 | −0.648382599 |
| 5 | 0.022355366 | −0.536165944 | −0.356965932 | −0.019461115 | 0.006575046 |
| 6 | −0.039517564 | 0.334080005 | 0.230837418 | −0.29585044 | −0.316875313 |
| 7 | −0.598181356 | −0.458893842 | −0.407181616 | 0.565739495 | 1.011139676 |
| 8 | −0.073366318 | −0.297729993 | −0.626464349 | 0.726932251 | 1.066673356 |
| 9 | −0.706118234 | −0.310629549 | 0.442551271 | 0.530107317 | 0.710743938 |

### DISCUSSION

In the past decade, various epigenetic clocks have been developed to predict chronological age, composite measures of biological age, single-cell epigenetic age, and aging in various tissues. Yet, what remained missing was a method to capture aging in different biological systems, independently and interactively, using a single blood test. While it was unclear to what extent signals in other organ systems could be captured in blood DNA methylation, our results suggest that this is possible. Systems Age is the first measure to capture heterogeneity in aging across different biological systems using epigenetic information from a single blood draw.

We showed that Systems Age scores were not only predictive of a wide variety of aging conditions and phenotypes at both baseline and follow-up, but were also specific to the pathophysiology of their intended system. For example, the Heart score was most associated with heart disorders CHD and MI, as well as overall mortality reflecting that cardiovascular disease is the leading cause of mortality worldwide. Heart was also strongly associated with thyroid disease, lung cancer, stroke, cataracts, reduced physical function, and total comorbidities, reflecting disease and treatment complications, shared risk factors, and shared pathophysiology. However, Heart demonstrated specificity in that it was only very weakly associated with diseases such as baseline arthritis or time-to-leukemia (instead these were most strongly associated with Musculoskeletal and Blood respectively). Likewise, the Inflammation score was strongly associated with time-to-CHD, baseline arthritis and baseline physical and cognitive functioning, which are expected based on known pathophysiology. Inflammation was the most strongly associated system with total number of comorbidities at baseline, consistent with inflammation driving many diseases of aging. The Brain score was associated with baseline cognitive functioning and time-to-stroke, but much less with most other phenotypes. The Musculoskeletal score was strongly associated with physical function and baseline arthritis as expected, as well as total comorbidities and baseline diabetes which can worsen musculoskeletal function, but was far less predictive of other phenotypes than other systems scores. Thus, blood DNA methylation data alone can be used to derive many different specific aging scores for various physiological systems, rather than just a single blood-specific or whole-body aging process.

We also observed unexpected results, such as negative associations of epigenetic aging with breast cancer risk in women, with the strongest negative association being Hormone. However, prior literature has shown that epigenetic clock acceleration is associated with a younger age at menopause (Levine, M. E. et al. Menopause accelerates biological aging. Proc. Natl. Acad. Sci. U.S.A 113, 9327-9332 (2016)). Simultaneously, later menopause has been linked to higher risk of reproductive organ cancers (Menarche, menopause, and breast cancer risk: individual participant meta-analysis, including 118 964 women with breast cancer from 117 epidemiological studies. Lancet Oncol. 13, 1141-1151 (2012); Trichopoulos, D., MacMahon, B. & Cole, P. Menopause and breast cancer risk. J. Natl. Cancer Inst. 48, 605-613 (1972)). Thus, women who epigenetically age faster may in fact have lower chances of getting reproductive organ cancer. It is also important to note that the two hormones being predicted by the Hormone score were DHEAS and IGF-1 both of which fall with age (Leng, S. X. et al. Serum levels of insulin-like growth factor-I (IGF-I) and dehydroepiandrosterone sulfate (DHEA-S), and their relationships with serum interleukin-6, in the geriatric syndrome of frailty. Aging Clin. Exp. Res. 16, 153-157 (2004)). Since Hormone score was negatively associated with both, higher Hormone score likely means lower DHEAS and IGF-1, which has been shown as protective for breast and endometrial cancer (Grimberg, A. Mechanisms by which IGF-I may promote cancer. Cancer Biol. Ther. 2, 630-635 (2003); Mahmud, K. Hormones and breast cancer: can we use them in ways that could reduce the risk? Oncol. Rev. 2, 146-153 (2008)).

Given the interconnectedness of aging, it was unclear prior to our study whether a given aging phenotype would be better predicted by epigenetic clocks that detected more global aging signals, or clocks trained on a limited set of clinical biomarkers specifically related to that phenotype. Our results demonstrated the advantages of the latter approach. Other recently developed clocks that strongly associated with specific aging conditions and phenotypes included PCPhenoAge (trained on a composite biological age measure that involves multiple systems), DNAmGrimAge (trained using smoking and proteins each involved in multiple systems), and DunedinPACE (trained on longitudinal changes across many systems). Thus, these clocks are intended to capture global aging signals that are not limited to any particular system. For 10 of the 14 phenotypes and diseases we tested, the most relevant system score surpassed all three of these clocks. While the relevant system score did not surpass the predictive ability of all the other clocks for other outcomes, the system scores were nearly as predictive, while being interpretable and granular-they reveal which systems are related to which phenotypes. Another advantage of system scores became apparent when looking across many phenotypes simultaneously. For example, DNAmGrimAge predicted mortality, cardiovascular outcomes, lung cancer, and thyroid dysfunction particularly well, but was less predictive of cognitive function, comorbidities, arthritis, diabetes, or leukemia risk. Interestingly, PCPhenoAge showed nearly the opposite pattern as DNAmGrimAge. DunedinPACE's pattern of association resembled a mixture between PCPhenoAge and DNAmGrimAge but was still not predictive of some phenotypes like thyroid dysfunction and leukemia risk. This suggested that epigenetic clocks that are directly trained to predict global proxies of aging can introduce biases in which aging phenotypes they are related to. In contrast, Systems Age showed more uniform prediction across all phenotypes, showing either the strongest or second-strongest associations with 12 of the 14 conditions, compared to the other three clocks. Thus, Systems Age appeared to not be strongly biased by a particular dimension of aging, which is likely the result of first training predictors of mortality in each physiological system independently before combining them. In further support of this idea, systems scores and Systems Age remain highly associated with all these phenotypes after correction for smoking.

In addition to heterogeneity at the systems level, we examined the heterogeneity that arose due to the interaction of different systems. Interestingly, some system scores were more correlated with each other than others. Heart and Lung were highly related, consistent with their common vulnerability to smoking, shared pulmonary circulation and combined function in oxygenating the body. The Liver and Blood scores showed strong correlations, potentially reflecting the liver's blood filtration function and production of blood products. Both Liver and Blood were highly correlated with Brain, potentially reflecting known contributions of anemia and altered levels of liver products to brain aging. Metabolic, Inflammation, and Kidney were highly correlated, reflecting numerous links between the metabolism and inflammation as well as the inclusion of IL-6 and CRP in both systems, and the contributions of both to kidney aging. The super-cluster involving Heart, Musculoskeletal, Liver, Blood, Brain, Metabolic, Inflammation, and Kidney can be similarly ascribed to numerous known interactions between systems as well as shared risk factors. Of note, while the correlations between systems do likely reflect true physiological interactions, it is also possible that some of the correlation structure can be attributed to similar mechanisms by which they impact the blood methylome and vice versa.

We defined subtypes as groups of individuals with similar systems specific aging scores that have an over or under representation of specific diseases and conditions. We showed the existence of 9 such distinct clusters or aging subtypes that had predisposition to very distinct diseases and conditions. Demonstration of the existence of aging subtypes has already been documented in the literature (Ahadi, S. et al. Personal aging markers and ageotypes revealed by deep longitudinal profiling. Nat. Med. 26, 83-90 (2020)). However, we provided evidence that these distinct groups can be observed even when using a single assay—in our case, DNA methylation assessed in blood. In the future, information on longitudinal changes will be critical for further defining age subtypes and their relevant disease risks. This could facilitate sub-classification of aging conditions and eventually inform targeted therapies based on aging subtypes.

Previous attempts have been made at capturing system-specific aging using data sources such as metabolomics, proteomic, clinical biomarkers, and other multi-Omics; however, the translatability of these models to clinic and wider application is a challenge given the limitations with usage of these data types in clinic due to technological limitations or harmonization across 100s of clinical biomarkers. Here we were able to accomplish a similar goal, while only relying on a single assay. Thus, the standardization offered by DNA methylation data generation allows for both potential wide-spread usage and ease of cross-comparison across experiments. Additionally, with recent developments, the decrease in cost of epigenetic data generation further shows its versatility as a superior source of biological information.

Systems Age as a framework for capturing heterogeneity shows a lot of promise, yet limitations remain. Due to lack of clinical measures for certain domains, we were not able to capture certain systems that are known to be affected by aging. Take for example, reproductive aging which has been shown to be a critical dimension of aging but was only captured indirectly in our framework. Clearly, there is a need for reproductive systems specific scores, something that can be developed in later iterations of such frameworks. Additionally, while we were able to approximate metabolic and hepatic aging, there is no score that shines light on aging in other digestive organ systems such as stomach, pancreas, colon and more. All these organ systems have distinct aging related conditions which are unfortunately not captured in the present scores. Similarly, sensory aging such as those pertaining to vision, hearing, and sensation are not captured in the system scores. All of these are potential future system scores that can be built into the framework. It is also important to note that one could add more clinical biomarkers and phenotypes to the systems, and there are other ways to map biomarkers to systems. Rather, it is unknown how robust these systems scores are to changes in the systems biomarkers used for training and thus there needs to be further testing done to shine light on these aspects. Another important future direction for Systems Age could be to detangle genetic predisposition to aging in certain systems as opposed to environmental effects on aging of systems. A very clear example of this is smoking, which leads to accelerated aging in certain systems and predisposition to specific aging related diseases. Conversely, there may also be genetic factors, which predispose certain systems to be more or less vulnerable than others (Kuo, C.-L., et al., Genetic associations for two biological age measures point to distinct aging phenotypes. Aging Cell 20, e13376 (2021)). Another caveat along the same lines is that it is unknown why DNAm in blood reflects aging in other physiological systems, and what is the molecular relationship between the clinical biomarkers, disease states and blood DNAm. It could reflect shared genetic variation, exposures, age-related patterns between tissues. Alternatively, it could involve intercellular signaling influencing DNAm in blood (either directly through epigenetic regulators or via changes in blood cell proportions), or blood DNAm reflecting processes by which immune cells affect aging in those systems. Further analysis needs to be performed to understand these relationships better. Finally, Systems Age uses only clinical data to first generate scores that are then predicted from epigenetic data. Other data types, such as proteomics, metabolomics, or imaging, may be highly informative when it comes to capture more diverse dimensions of aging.

Overall, we highlight the importance of capturing heterogeneity in aging while also building a reusable framework for quantifying multifactorial aging phenotypes. We show that this level of dimensionality can be estimated from a single data source—in this case DNA methylation in blood. The scores built using our approach perform as well, or in many cases better than, presently available epigenetic clocks, while simultaneously providing the potential to identify individuals with distinct aging subtypes for clinical healthcare and drug development purposes.

### Methods

### Datasets Used for Training Systems Age

Two different longitudinal studies were used for training Systems Age: the Health and Retirement Study (HRS) and Framingham Heart Study (FHS) (Table 12). We previously utilized and described methylation data from these datasets in a separate study (Higgins-Chen et al., A Computational Solution for Bolstering Reliability of Epigenetic Clocks: Implications for Clinical Trials and Longitudinal Tracking. Nat Aging 2, 644-661 (2022)). Briefly, HRS is a nationally representative sample of Americans over age 50 years, with data available to qualified academic researchers, requiring an application at hrs.isr.umich.edu. HRS had biomarker information available for 9,933 participants of which Infinium Methylation EPIC BeadChip data was available for 4,018 individuals (Crimmins, E. M., et al., Associations of Age, Sex, Race/ethnicity, and Education with 13 Epigenetic Clocks in a Nationally Representative US Sample: The Health and Retirement Study. J Gerontol A Biol Sci Med Sci. 76 (6): 1117-1123 (2021 May 22).). Out of the 4018 individuals only 3,593 had clinical data (age range 51-100 years) which were used for training of Systems Age. The study was approved by the Institutional Review Board (IRB) at the University of Michigan (HUM00061128). All participants provided written informed consent.

FHS includes 2,748 FHS Offspring cohort participants attending the eighth exam cycle (2005-2008) and 1,457 Third Generation cohort participants attending the second exam cycle (2005-2008), who consented to provide their DNA for genomic research (Kannel et al., An Investigation of Coronary Heart Disease in Families: The Framingham Offspring Study.” American Journal of Epidemiology 110 (3): 281-90 (1979); Splansky et al., The Third Generation Cohort of the National Heart, Lung, and Blood Institute's Framingham Heart Study: Design, Recruitment, and Initial Examination. American Journal of Epidemiology 165 (11): 1328-35 (2007)). DNA methylation was assayed with the Infinium HumanMethylation 450 BeadChip and is available in dbGaP (accession no. phs000724.v7.p11) to qualified academic researchers, requiring an application. For the purpose of training Systems Age, FHS Offspring data was used but for scaling of system scores and systems age to age range both the Offspring and Third generation data was used. Deaths of FHS participants occurring before 1 Jan. 2014 were ascertained by contact with participants, hospital surveillance, local obituaries and the National Death Index. The study protocol was approved by the IRB at Boston University Medical Center. All participants provided written informed consent at the time of each examination visit.

TABLE 12
Datasets used for training with total number of samples, female
percentage, age distribution, death, and follow-up years.

| | Total number | | | Deaths and |
|---|---|---|---|---|
| Dataset | of samples | Female | Age | follow-up |
| HRS | 9,933 | 52% | 63.1 ± 14.4 | NA |
| biomarkers | | | | |
| HRS | 4018 | 41% | 69.6 ± 9.7 | NA |
| methylation | | | | |
| FHS | 3935 | 47% | 58.49 ± 13.2 | 319 with |
| methylation | | | | 6.8 ± |
| | | | | 2.2 years |

### Systems Age Pipeline

Step 1: Grouping Biomarkers into Systems

We utilized molecular and cellular biomarker data from the Health and Retirement Study (HRS) 2016 Venous Blood Study (VBS), for which a subset also has paired DNA methylation data. We assessed the available biomarkers, and manually annotated them as biomarkers for specific physiological systems, totaling 11 systems. To each system we added functional biomarkers (e.g. grip strength) and system-specific disease and condition history (e.g. history of stroke or chronic lung disease).

Our goal was to develop epigenetic aging clocks that are interpretable in terms of physiological systems for clinical and epidemiological applications. Thus, for manual annotation, we required biomarkers to fulfill at least one of two criteria to be assigned to a system: 1) Is there evidence that the biomarkers predict risk of age-related diseases for that physiological system? 2) Would a clinician utilize the biomarker in assessing the status of that physiological system? Annotations were done by multiple team members supported by literature searches to validate disease prediction and clinical interpretations. Most of the biomarkers were transformed and thresholded such that their distribution is more normal. The biomarker-to-system mapping, dataset-specific variable names, and transformations used can be found in Table 2.

There is no gold standard list of biomarkers for each physiological system and there is often not a clear delineation between systems because of their biological integration. We do not claim that these are the only 11 systems or the only correct mapping of the Biomarkers to these 11 systems. The Systems Age pipeline can be easily adapted to other biomarker-to-system mappings. Our work here is intended as a proof-of-concept that omics clocks can capture aging in specific physiological systems, and thus the most important validation of our chosen mappings is the high specificity of the System scores in our WHI validation dataset, rather than the exact list of starting biomarkers.

### Step 2: Principal Component Analysis of System Biomarkers and DNA Methylation Data

We previously found that performing principal component analysis (PCA) and then using principal components (PCs) as input into supervised machine learning models produces more robust and reliable epigenetic clocks (Higgins-Chen et al. A computational solution for bolstering reliability of epigenetic clocks: implications for clinical trials and longitudinal tracking. Nat Aging 2, 644-661 (2022)). PCA removes collinearity, reduces dimensionality of the data, and better separates signal from technical noise. Thus, for each system, we performed PCA on the selected system biomarkers. Before performing PCA, the biomarkers were first transformed to have a normal distribution as described in Table 2 as well as scaled before inputting into the prcomp function (stats 4.1.1) in R. In parallel, we performed PCA on DNA methylation data as previously described (Higgins-Chen et al., A computational solution for bolstering reliability of epigenetic clocks: implications for clinical trials and longitudinal tracking. Nat Aging 2, 644-661 (2022)), utilizing 125,175 CpGs that 1) are in all of our training and validation data and 2) present on commercially available methylation arrays including the Infinium HumanMethylation 450 BeadChip and Infinium Methylation EPIC BeadChip. Practically, this was done using the prcomp function in R. This yielded two sets of PCs: 1) system biomarker PCs (the number of PCs per system is equivalent to the number of biomarkers for each system, since number of samples is greater than number of features) and 2) 4,017 DNA methylation PCs (one less than number of samples, since the number of samples is less than number of features). See Table 13 for terminology. We did not filter out low-variance PCs (for example using scree plots or random matrix theory methods). Low-variance PCs can still capture relevant variation for prediction, while those that are irrelevant are removed or minimized at later supervised machine learning steps. Thus, when predicting system biomarker PCs from DNA methylation PCs (Step 3), we can predict both dominant, shared signals between biomarkers (high-variance PCs) as well as more subtle variations.

### Step 3: Building DNAm Surrogates of System PCs

We utilize elastic net regression to train a model using methylation PCs to predict each system biomarker PC using the glmnet 4.1-4 package in R. We refer to the resulting models as DNAm system PCs. This was done as described previously (Higgins-Chen et al. 2022). The L1 to L2 regularization ratio was 1 (α=0.5), the λ tuning parameter was selected via tenfold cross-validation, and the final methylation PC was excluded as it is not meaningful in cases where the number of samples is less than the number of features. Not all system PCs are predicted well using methylation PCs. We retained DNAm system PCs with at least 20 DNAm PCs being used at the minimum mean cross-validated error in the model and at least 5 DNAm PCs at the cross-validated error one standard error from the minimum mean cross-validated error in the model. This allows us to take only well predicted DNAm system PCs to the next step.

### Step 4: Building System Scores

To build system scores we first calculate DNAm system PCs in FHS based on parameters previously trained in HRS (first calculating methylation PCs, then predicting system PCs). Then, for each system separately, we predicted mortality using DNAm system PCs in a Cox elastic net mortality prediction model using the glmnet 4.1-4 package. The L1 to L2 regularization ratio was 1 (α=0.5), the λ tuning parameter was selected via tenfold cross-validation. This yielded 11 separate mortality prediction models that we term system scores, and can serve as a measure of mortality-related deterioration of each system.

### Step 5: Building Systems Age

To build Systems Age, we first perform PCA on the DNAm system scores and age prediction score using the prcomp (stats 4.1.1) function in R, as the system scores and age prediction score are partially correlated with one another.

The age prediction score is built specifically to predict chronological age and was trained in HRS. The DNAm PCs in HRS were first used to predict chronological age. The scores thus generated were then used to predict chronological age again but instead now using a second degree polynomial function fitted to the 5 year interval averages of the predicted chronological age score (previous step) predicting for the 5 year interval averages of chronological age. The score obtained from the second degree polynomial is referred to as age prediction in our model.

Using all system score PCs, we then predict mortality using another Cox elastic net mortality prediction model using glmnet 4.1-4 package in R. The L1 to L2 regularization ratio was 1 (α=0.5), the λ tuning parameter was selected via tenfold cross-validation, Again, using PCs as input is intended to reduce redundancy, increase reliability, and allow for more subtle variations in system scores to have an important role in the overall model.

### Step 6: Scaling Scores to Age Range

The 11 system scores and Systems Age are first standardized to have mean 0 and standard deviation 1. They are then scaled to match the mean and standard deviation of chronological age for the 3935 samples from FHS Offspring and Gen3 cohorts.

TABLE 13
Terms used to describe Systems Age and intermediate
values derived during Systems Age calculation

| Term | Definition |
|---|---|
| Methylation PCs | Principal components generated by |
| | performing PCA on methylation data with |
| | 125K CpGs from HRS |
| System biomarker PCs | Principal components generated by |
| | performing PCA on groups of Biomarkers in |
| | HRS |
| DNAm system PCs | Methylation PC based surrogates of system |
| | biomarker PCs |
| System scores | Cox elastic net regression model scores |
| | generated by combining DNAm system PCs |
| | for each system |
| System score PCs | Principal components generated by |
| | performing PCA on the 11 system scores plus |
| | a chronological age prediction score in FHS |
| Systems Age | Whole body aging score generated by |
| | combining system score PCs using a cox |
| | elastic net regression model |

### Association Meta-Analysis in WHI Cohorts

The Women's Health Initiative (WHI) is a long-term national health study (The Women's Health Initiative Study Group, Design of the Women's Health Initiative Clinical Trial and Observational Study. Controlled Clinical Trials 19 (1): 61-109 (1998)) WHI is funded by the National Heart, Lung, and Blood Institute, or NHLB and ran from the early 1990s to 2005. Post 2005, there have been Extension Studies, which continue to collect data on health outcomes annually. We used 3 WHI cohorts which had methylation data available. In each WHI cohort (Table 14) we calculated system scores then regressed all epigenetic aging clocks on chronological age using a linear regression model and defined clock age acceleration as the corresponding residual. We then calculated associations between these clock accelerations and different diseases and aging phenotypes in all WHI cohorts. We stratified the cohorts by race (except WHI AS311 where analysis of the Black and Hispanic populations would be underpowered), for a total of 7 groups. Depending on condition and disease we either built linear regression models (cognitive function, physical function, comorbidities and more), cox prediction models (Lung Cancer, Breast Cancer, Leukemia, CHD, MI and more) or logistic regression models (Thyroid disease, Diabetes, Arthritis, Cataract and more) to look at associations with the age accelerated scores. An example of the formula used is as follows: Cognitive function~AgeAccel+Age. In certain cases, additional factors such as Education level (for Cognitive function) were also added to the models. Sex was not a covariate as all WHI participants are female. We combined the associations from the different cohorts and racial groups in a fixed effects model meta-analysis with inverse variance weights, obtaining meta-analysis Z-scores for the associations. Forest plots, z-scores, heterogeneity p-values and other meta-analysis results are provided in FIG. 7 and Table 4.

TABLE 14
WHI cohorts used for testing with racial distribution,
percent current and percent past smokers

| WHI | Number of | | | | Current | Past |
|---|---|---|---|---|---|---|
| cohort | samples | White | Black | Hispanic | Smokers | smokers |
| BAA23 | 2107 | 998 | 676 | 433 | 213 | 756 |
| AS311 | 855 | 760 | 70 | 25 | 73 | 393 |
| EMPC | 2167 | 1207 | 606 | 354 | 181 | 840 |

We performed additional analyses adjusting for smoking status by adding smoking status (present smoker, ex-smoker or never smoked) into the linear, Cox and logistic models. We also examined non-smokers separately. A list of the variables used from WHI are shown in Table 15. It is important to note that even though multiple disease variables were available we could not test for a majority of the variables because they were underpowered. Rather, for many of the variables we calculated z-scores and showed them in our supplementary data. Variables which had insufficient N have not been listed below. (Table 15)

TABLE 15
WHI variables used for testing associations of scores

| Condition | Variable used | Available in | Additional notes |
|---|---|---|---|
| Cognitive | 100 - F393MSE | Form 39 - Cognitive | F393MSE score |
| Function | | Assessment | mapped using the |
| | | | year at which blood |
| | | | was drawn for |
| | | | methylation |
| Physical | 100 - PHYSFUN | Form 38 - Daily Life | PHYSFUN score |
| function | | | mapped using the |
| | | | year at which blood |
| | | | was drawn for |
| | | | methylation |
| Mortality | DEATHALL, | 2021 Oct. 7 | Corrected for time to |
| | ENDFOLLOWALLDY | Outcomes | date at which blood |
| | | | for methylation was |
| | | | drawn |
| CHD | CHD, CHDDY | 2021 Oct. 7 | Corrected for time to |
| | | Outcomes | date at which blood |
| | | | for methylation was |
| | | | drawn |
| MI | MI, MIDY | 2021 Oct. 7 | Corrected for time to |
| | | Outcomes | date at which blood |
| | | | for methylation was |
| | | | drawn |
| Lung Cancer | LUNG, LUNGDY | 2021 Oct. 7 | Corrected for time to |
| | | Outcomes | date at which blood |
| | | | for methylation was |
| | | | drawn |
| Breast Cancer | BREAST, BREASTDY | 2021 Oct. 7 | Corrected for time to |
| | | Outcomes | date at which blood |
| | | | for methylation was |
| | | | drawn |
| Endometrial | ENDMTRL, | 2021 Oct. 7 | Corrected for time to |
| Cancer | ENDMTRLDY | Outcomes | date at which blood |
| | | | for methylation was |
| | | | drawn |
| Leukemia | LEUKEMIA, | 2021 Oct. 7 | Corrected for time to |
| | LEUKEMIADY | Outcomes | date at which blood |
| | | | for methylation was |
| | | | drawn |
| Skin Cancer | MELANOMA, | 2021 Oct. 7 | Corrected for time to |
| | MELANOMADY | Outcomes | date at which blood |
| | | | for methylation was |
| | | | drawn |
| Colorectal | COLORECTAL, | 2021 Oct. 7 | Corrected for time to |
| Cancer | COLORECTALDY | Outcomes | date at which blood |
| | | | for methylation was |
| | | | drawn |
| Stroke | STROKE, | 2021 Oct. 7 | Corrected for time to |
| | STROKEDY | Outcomes | date at which blood |
| | | | for methylation was |
| | | | drawn |
| Thyroid | THYROID | Form 30 Medical | At baseline |
| Disease | | History | |
| Cataract | CATARACT | Form 30 Medical | At baseline |
| | | History | |
| Arthritis | ARTHRIT | Form 30 Medical | At baseline |
| | | History | |
| Diabetes | DIAB | Form 30 Medical | At baseline |
| | | History | |
| Any condition | NACOND | Form 30 Medical | At baseline |
| | | History | |
| Total | Calculated using all | Form 30 Medical | At baseline |
| Comorbidities | available variables in Form | History | |
| | 30 such as CVDEVER, | | |
| | DIAB, ALS, ARTHRIT, | | |
| | THYROID, CANC_F30 | | |
| | and more | | |
| Smoking status | SMOKING | Form 34 - Personal | |
| | | Habits | |
| Age | AGE | Demographics and | Corrected for time to |
| | | Study Membership | date at which blood |
| | | | for methylation was |
| | | | drawn |

### Calculating Different Clocks

In addition to Systems Age, we calculated a large number of additional existing clocks for comparison. We used the following packages or sources to do so (Table 16).

TABLE 16
Packages used for analysis

| Clock | Package/Source |
|---|---|
| PC clocks | PC-clocks R package - |
| | https://github.com/MorganLevineLab/PC-Clocks |
| Methylation clocks except | methylCIPHER R package - |
| DunedinPACE and GrimAge | https://github.com/MorganLevineLab/methylCIPHER |
| DunedinPACE | DunedinPACE R package - |
| | https://github.com/danbelsky/DunedinPACE |
| GrimAge | Pre-calculated values as provided on the |
| | WHI server |

### Aging Subtypes and Overrepresentation of Diseases in Subtypes

Age-adjusted system scores were used to perform adaptive hierarchical clustering using the Dynamic Tree Cut library (dynamicTreeCut 1.63-1, function cutreeDynamic Tree) in R. Parameters used other than default settings included minModuleSize which was set at 100. Based on the most stable node distance, 9 clusters were identified. Average score for each system for each cluster was plotted on polar spider plots. An over representation analysis comparing occurrence of disease in the cluster compared to the whole population was performed using Fisher's exact test. Binary disease status variables were used without transformation, continuous variables such as cognitive function and physical function were converted into binary variables by marking values lesser than 1 standard deviation from mean as disease states. For time-to-event variables, the model was built only for individuals who were alive until the 7 year follow-up or died because of the condition.

### Test-Retest Reliability Analysis

Reliability was calculated as described before (Higgins-Chen et al. 2022). Briefly, reliability was calculated in GSE55763 which consisted of 36 whole-blood samples measured in duplicate (age range 37.3 to 74.6). We used the icc function in the irr R package version 0.84.1, using a single-rater, absolute-agreement, two-way random-effects model.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

What is claimed is:

1. A method comprising:
(a) obtaining a biological sample from a subject;
(b) deriving one or more nucleic acid molecules from the biological sample of the subject; and
(c) sequencing the one or more nucleic acid molecules using a single genomic sequencing assay.

2. The method of claim 1, wherein the one or more nucleic acid molecules comprise deoxyribonucleic acid (DNA).

3. The method of claim 2, wherein the sequencing further comprises sequencing the DNA using the single genomic sequencing assay.

4. The method of claim 3, wherein the single genomic sequencing assay comprises a DNA sequencing assay.

5. The method of claim 4, wherein the DNA sequencing assay is a methylation sequencing assay.

6. The method of claim 5, further comprising sequencing the DNA using the methylation sequencing assay.

7. The method of claim 6, further comprising sequencing a methylome of the DNA using the methylation sequencing assay.

8. The method of claim 1, wherein the biological sample is obtained from the subject at a single sampling time point.

9. The method of claim 1, wherein the biological sample of the subject comprises a blood sample of the subject.

10. The method of claim 9, wherein the blood sample of the subject is obtained from the subject at a single blood draw.

11. The method of claim 10, wherein the blood sample of the subject is a single blood sample obtained from the subject at the single blood draw.

12. The method of claim 9, wherein the blood sample of the subject is a whole blood sample.

13. The method of claim 9, wherein the blood sample of the subject is a blood plasma sample or a blood serum sample of the subject.

14. The method of claim 13, wherein the blood plasma sample is obtained or derived from the blood sample of the subject.

15. The method of claim 13, wherein the blood serum sample is obtained or derived from the blood sample of the subject.

16. The method of claim 2, further comprising fragmenting the DNA, producing a plurality of DNA fragments.

17. The method of claim 16, further comprising sequencing at least a subset of the plurality of DNA fragments using the single genomic sequencing assay.

18. The method of claim 17, wherein the single genomic sequencing assay comprises a methylation sequencing assay.

19. The method of claim 18, further comprising sequencing a methylome of a DNA fragment of the plurality of DNA fragments.

20. The method of claim 1, wherein the subject is a human subject.

21. The method of claim 20, wherein the subject is a human subject having cancer.

22. The method of claim 20, wherein the subject is an adult human subject.

23. The method of claim 22, wherein the adult human subject is over 18 years of age.

24. The method of claim 22, wherein the adult human subject is over 50 years of age.

25. The method of claim 1, wherein the biological sample is a saliva sample.
